(19) United States
(12) Patent Application Publication
(10) Pub. No.: US 2024/0042038 A1
(43) Pub. Date: Feb. 8, 2024

(54) FORMULATIONS OF CABAZITAXEL

(71) Applicant: Zhuhai Beihai Biotech Co., Ltd., Jinwan (CN)

(72) Inventor: Qun SUN, Princeton, NJ (US)

(21) Appl. No.: 18/044,309

(22) PCT Filed: Sep. 13, 2021
(86) PCT No.: PCT/US2021/050091

§ 371 Date: Mar. 7, 2023

Related U.S. Application Data
(60) Provisional application No. 63/241,999, filed on Sep. 8, 2021.
(60) Provisional application No. 63/227,180, filed on Jul. 29, 2021.
(60) Provisional application No. 63/078,067, filed on Sep. 14, 2020.

(51) Int. Cl. A61K 47/42; A61K 9/00; A61K 31/337; A61K 47/10; A61J 1/10; A61J 1/20

(52) U.S. Cl. CPC A61K 47/42; A61K 9/0019; A61K 31/337; A61K 47/10; A61J 1/10; A61J 1/20

(57) ABSTRACT

This document relates to a pharmaceutical formulation of cabazitaxel with two compositions that are mixed prior to being infused or administered to patients, that comprises: (a) a first liquid composition comprising cabazitaxel and ethanol, and (b) a second aqueous composition comprising human serum albumin and a parenterally acceptable vehicle, and wherein the said pharmaceutical formulation does not contain a surfactant (e.g. Polysorbate 80).

### CLAIM OF PRIORITY

This application claims priority to U.S. Provisional Patent Application Ser. No. 63/078,067, filed on Sep. 14, 2020, U.S. Provisional Patent Application Ser. No. 63/227,180, filed on Jul. 29, 2021, and U.S. Provisional Patent Application Ser. No. 63/241,999, filed on Sep. 8, 2021 the entire contents of which are hereby incorporated by reference.

### TECHNICAL FIELD

This document relates to formulations for the treatment of proliferative diseases, more particularly to formulations comprising Cabazitaxel.

### BACKGROUND

Many drugs for parenteral use are insoluble in water, and are thus formulated with solubilizing agents, surfactants, solvents, and/or emulsifiers that are irritating, allergenic, or toxic when administered to patients. See, e.g., Briggs et al., Anesthesis 37, 1099 (1982), and Waugh et al., Am. J. Hosp. Pharmacists, 48, 1520 (1991)). Further, many of these drugs, especially those administered intravenously, cause undesirable side effects such as venous irritation, phlebitis, burning and pain on injection, venous thrombosis, extravasation, and other administration related side effects. Additionally, often free drugs present in formulations induce pain or irritation upon administration.

Taxanes play an important role in the treatment of various solid tumors. Cabazitaxel (trade name Jevtana®) is a semi-synthetic taxane derivative. It was developed by Sanofi-Aventis and was approved by the U.S. FDA for the treatment of hormone-refractory prostate cancer on Jun. 17, 2010. Cabazitaxel in combination with prednisone is a treatment option for hormone-refractory prostate cancer following cabazitaxel-based treatment. JEVTANA is supplied as a kit consisting of (a) a JEVTANA injection, which contains 60 mg cabazitaxel in 1.5 mL polysorbate 80; and (b) a diluent, containing approximately 5.7 mL 13% (W/W) ethanol. Prior to administration, the JEVTANA injection must first be mixed with the diluent, which dilutes the amount of Cabazitaxel to 10 mg/mL, and then further diluted with either 0.9% sodium chloride solution or 5% dextrose solution for infusion. See JEVTANA Prescribing Information.

Other taxane compounds include docetaxel, which is marketed as Taxotere® and is FDA-approved for breast cancer, non-small cell lung cancer, hormone refractory prostate cancer, gastric adenocarcinoma, and squamous cell carcinoma of head and neck cancer. The clinical intravenous administration of commercially available docetaxel (Taxotere®) is formulated in a highly concentrated solution containing 40 mg docetaxel and 1040 mg Polysorbate 80 per mL. See TAXOTERE Prescribing Information.

The presence of polysorbate 80 in JEVTANA, as well as TAXOTERE, can result in serious side effects. It has been reported that docetaxel administration is associated with the occurrence of unpredictable (acute) hypersensitivity reactions and cumulative fluid retention. See, e.g., Trudeau M E et al., J Clin Oncol 1996; 14:422-8, Piccart M J et al., J Natl Cancer Inst 1995; 87:676-81, Bruno R et al., J Clin Oncol 1998; 16:187-96. These side-effects have been attributed, in part, to the presence of polysorbate 80.

In order to reduce the side effects induced by polysorbate 80, patients may be treated with dexamethasone prior to each dose of JEVTANA. Dexamethasone is a steroid that suppresses the immune response in patients, which can be especially detrimental in cancer patients under chemotherapy, whose immunity may already be compromised due to the destruction of healthy cells by the chemotherapeutic treatment. As a result, these patients can be susceptible to bacterial and fungal infections. Further, despite receiving the dexamethasone pre-medication, patients can report hypersensitivity side effects from the taxane compound treatment. Due to these side effects, patients may stop taxane compound therapy, skip a dose, or continue further therapy at a reduced dose.

New formulations of cabazitaxel without polysorbate 80 have been reported. WO2017/123760 and WO2019/204738 describes compositions and formulations including cabazitaxel and human serum albumin. China patent applications CN104490797A, CN104224750A, CN103393632A, CN106852911A, and CN105727303A describes nanoparticle compositions of cabazitaxel and albumin.

Novel formulations of cabazitaxel are further needed to avoid these side effects, pre-medication requirements, and patient noncompliance issues associated with the currently marketed formulation, and yet are more convenient to use in clinic.

### SUMMARY

The current marketed formulation of Jevtana comprises a two-vial formulation (injection and diluent). The two-vial formulation contains a solution of cabazitaxel in polysorbate 80 and a solvent vial containing an aqueous solution of 13% ethanol (w/w).

The two-vial formulation requires two dilutions prior to admission to the patient. In the two-vial formulation, the drug vial prior to use has to be reconstituted with the solvent vial making sure that polysorbate 80 is properly reconstituted without significant foaming. The reconstituted solution is further diluted by injection of the appropriate amount of the solution into an infusion bag. Thus, for the two-vial formulation there is risk of foaming and not convenient to use. Furthermore, the current marketed formulation contains polysorbate 80, which could cause severe hypersensitivity reactions to some patients. There is a black box warning for hypersensitivity reactions with polysorbate 80 on the product label for Jevtana. It would be highly desirable to develop alternative cabazitaxel formulations which do not contain polysorbate 80 and yet are convenient to use in clinic.

In present invention, the applicant has invented a novel formulation which contains the drug product as a solution in a single vial with known concentration. The drug product solution is withdrawn from the vial and injected into an infusion bag/bottle prior to use. The vial does not require to be reconstituted and homogenized prior to use. Thus, for this novel formulation there is no risk of foaming and the formulation is more convenient to use. In addition, the novel formulation does not contain polysorbate 80. The applicant has surprisingly found that by adding a solution of human serum albumin into an infusion bag or bottle with a parenterally acceptable vehicle (e.g., a normal saline solution, a dextrose solution, etc.) prior to injecting the drug product solution into the infusion bag, there is no need to use polysorbate 80 in the cabazitaxel formulation of the present disclosure.

In present invention, provided herein is a pharmaceutical formulation of cabazitaxel comprising two compositions that are mixed prior to being infused or administered to patients, that comprises: (a) a first liquid composition comprising cabazitaxel and ethanol, wherein the cabazitaxel is dissolved in a solvent comprising ethanol, and (b) a second aqueous composition comprising human serum albumin and a parenterally acceptable vehicle, and wherein the said pharmaceutical formulation does not contain a surfactant (e.g. Polysorbate 80). In some embodiments, the two compositions are mixed in less than or equal to about 24 hours prior to being infused or administered to patients. In some embodiments, the two compositions are mixed in less than or equal to about 8 hours prior to being infused or administered to patients. In some embodiments, the two compositions are mixed in less than or equal to about 6 hours prior to being infused or administered to patients. In some embodiments, the two compositions are mixed in less than or equal to about 2 hours prior to being infused or administered to patients. In some embodiments, the two compositions are mixed in less than or equal to about 1 hour prior to being infused or administered to patients. In some embodiments, the mixing is done in an infusion bag or infusion bottle. In some embodiments, the second aqueous composition is contained in an infusion bag or infusion bottle. In some embodiments, the first liquid composition is injected into an infusion bag or infusion bottle which contains the second aqueous composition.

In some embodiments, the said pharmaceutical formulation does not comprise a lipid (e.g. soybean oil). In some embodiments, the first liquid composition does not comprise a lipid (e.g. soybean oil). In some embodiments, the second aqueous composition does not comprise a lipid (e.g. soybean oil). In some embodiments, the lipid is soybean oil.

In some embodiments, the first liquid composition does not comprise a surfactant. In some embodiments, the second aqueous composition does not comprise a surfactant.

In some embodiments, the first liquid composition comprises cabazitaxel, ethanol, and other organic solvents (e.g. propylene glycol, polyethylene glycol, hereinafter “PEG”, etc.). The PEG preferably has a molecular weight of about 300, i.e. PEG 300, or about 400, i.e. PEG 400. Other molecular weight PEG's known to those of ordinary skill can be included if desired in alternative embodiments. In some embodiments, the first liquid composition comprises cabazitaxel, ethanol, and propylene glycol. In some embodiments, the first liquid composition comprises cabazitaxel, ethanol, and PEG. In some embodiments, the first liquid composition comprises cabazitaxel, ethanol, and PEG 300. In some embodiments, the first liquid composition comprises cabazitaxel, ethanol, and PEG400. In some embodiments, the first liquid composition further comprises an acid. In some embodiments, the first liquid composition comprises cabazitaxel, ethanol, and an organic acid. The most preferred acid for use in accordance with the present invention is citric acid. In some embodiments, the first liquid composition comprises cabazitaxel, ethanol, and citric acid. In some embodiments, the first liquid composition comprises cabazitaxel, ethanol, propylene glycol, and citric acid. In some embodiments, the first liquid composition comprises cabazitaxel, ethanol, and citric acid. In some embodiments, the first liquid composition comprises cabazitaxel, ethanol, PEG (e.g. PEG 300, PEG 400), and citric acid. In some embodiments, the weight ratio of cabazitaxel and citric acid in the first liquid composition is from about 5000:1 to about 1:1. In some embodiments, the weight ratio of cabazitaxel and citric acid in the first liquid composition is from about 2000:1 to about 1:1. In some embodiments, the weight ratio of cabazitaxel and citric acid in the first liquid composition is from about 1000:1 to about 1:1. In some embodiments, the weight ratio of cabazitaxel and citric acid in the first liquid composition is from about 500:1 to about 1:1. In some embodiments, the weight ratio of cabazitaxel and citric acid in the first liquid composition is from about 5000:1 to about 10:1. In some embodiments, the weight ratio of cabazitaxel and citric acid in the first liquid composition is from about 2000:1 to about 10:1. In some embodiments, the weight ratio of cabazitaxel and citric acid in the first liquid composition is from about 1000:1 to about 10:1. In some embodiments, the weight ratio of cabazitaxel and citric acid in the first liquid composition is from about 1000:1 to about 100:1. In some embodiments, the weight ratio of cabazitaxel and citric acid in the first liquid composition is from about 600:1 to about 200:1.

In some embodiments, the first liquid composition comprises cabazitaxel and a solvent including ethanol, wherein the cabazitaxel is dissolved in the solvent (e.g., the first liquid composition is a homogenous solution of cabazitaxel in ethanol). In some embodiments, the first liquid composition comprises cabazitaxel, an acid, and a solvent including ethanol, wherein the cabazitaxel and the acid are dissolved in the solvent. In some embodiments, the first liquid composition comprises cabazitaxel, an organic acid, and a solvent including ethanol, wherein the cabazitaxel and the organic acid are dissolved in the solvent. In some embodiments, the first liquid composition comprises cabazitaxel, citric acid, and a solvent including ethanol, wherein the cabazitaxel and citric acid are dissolved in the solvent. In some embodiments, the first liquid composition comprises cabazitaxel and ethanol, wherein the cabazitaxel is dissolved in ethanol. In some embodiments, the first liquid composition comprises cabazitaxel, citric acid, and ethanol, wherein the cabazitaxel and citric acid are dissolved in ethanol. In some embodiments, the first liquid composition comprises cabazitaxel, and dehydrated ethanol, wherein the cabazitaxel is dissolved in dehydrated ethanol. In some embodiments, the first liquid composition comprises cabazitaxel, citric acid, and dehydrated ethanol, wherein the cabazitaxel, and citric acid are dissolved in dehydrated ethanol.

In some embodiments, the first liquid composition is prepared by dissolving cabazitaxel in a solvent comprising ethanol. In some embodiments, the first liquid composition is prepared by dissolving cabazitaxel and citric acid in a solvent comprising ethanol. In some embodiments, the first liquid composition is prepared by dissolving cabazitaxel and citric acid in a solvent comprising ethanol and propylene glycol. In some embodiments, the first liquid composition is prepared by dissolving cabazitaxel and citric acid in a solvent comprising ethanol and PEG (e.g., PEG 300, PEG 400).

In some embodiments, the second aqueous composition is prepared by adding a human serum albumin solution into a parenterally acceptable vehicle. In some embodiments, the second aqueous composition is prepared by adding a human serum albumin solution into an infusion bag or bottle which contains a parenterally acceptable vehicle. In some embodiments, the second aqueous composition is prepared by adding a human serum albumin solution into a normal saline solution or dextrose solution. In some embodiments, the second aqueous composition is prepared by adding a human serum albumin solution into an infusion bag or bottle which contains a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a 20% solution of human serum albumin for infusion (w/v) into a normal saline solution or dextrose solution. In some embodiments, the second aqueous composition is prepared by adding a 25% solution of human serum albumin for infusion (w/v) into a normal saline solution or dextrose solution. In some embodiments, the second aqueous composition is prepared by adding a 5% solution of human serum albumin for infusion (w/v) into a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a 20% solution of human serum albumin for infusion (w/v) into an infusion bag or bottle which contains a normal saline solution or dextrose solution. In some embodiments, the second aqueous composition is prepared by adding a 25% solution of human serum albumin for infusion (w/v) into an infusion bag or bottle which contains a normal saline solution or dextrose solution. In some embodiments, the second aqueous composition is prepared by adding a 5% solution of human serum albumin for infusion (w/v) into an infusion bag or bottle which contains a normal saline solution or dextrose solution. In some embodiments, the second aqueous composition is an aqueous solution comprising human serum albumin. In some embodiments, the concentration of human serum albumin in the second aqueous composition is from about 0.1% to about 20% (w/v). In some embodiments, the concentration of human serum albumin in the second aqueous composition is from about 0.1% to about 10% (w/v). In some embodiments, the concentration of human serum albumin in the second aqueous composition is from 0.10% to 2% (w/v). In some embodiments, the concentration of human serum albumin in the second aqueous composition is from 0.2% to 5% (w/v). In some embodiments, the concentration of human serum albumin in the second aqueous composition is from 0.3% to 2% (w/v). In some embodiments, the concentration of human serum albumin in the second aqueous composition is from 0.3% to 1.5% (w/v). In some embodiments, the second aqueous composition comprises from about 0.1 g to about 20 g of human serum albumin. In some embodiments, the second aqueous composition comprises from about 0.5 g to about 10 g of human serum albumin.

Also, provided herein is a transparent parenteral infusion composition of cabazitaxel free of visible particles and precipitates comprising human serum albumin and said cabazitaxel in a concentration of cabazitaxel from about 0.01 mg/ml to about 0.5 mg/ml in a parenterally acceptable vehicle, wherein the said parenteral infusion solution is obtained by injecting a first liquid composition comprising cabazitaxel and ethanol into an infusion bag or bottle containing a second aqueous composition comprising human serum albumin in a parenterally acceptable vehicle, wherein mixing or agitation is not needed in the injection process. In some embodiments, the said cabazitaxel infusion solution does not contain a surfactant (e.g. Polysorbate 80). In some embodiments, a parenterally acceptable vehicle is a normal saline solution or dextrose solution.

Also, provided herein is a transparent parenteral infusion composition of cabazitaxel free of precipitates comprising human serum albumin, said cabazitaxel, and ethanol, wherein concentration of cabazitaxel in said infusion composition is from about 0.05 mg/ml to about 0.2 mg/ml, concentration of ethanol in the infusion composition if from about 0.2 vol. % to about 4 vol. %, and weight ratio of human serum albumin to cabazitaxel is from about 10:1 to about 300:1. Said parenteral infusion solution may be obtained by injecting a first liquid composition comprising cabazitaxel and ethanol into an infusion bag or bottle containing a second aqueous composition comprising human serum albumin in a parenterally acceptable vehicle. In some embodiments, mixing or agitation is not needed in the injection process. In some embodiments, he said cabazitaxel infusion solution does not contain a surfactant (e.g. Polysorbate 80).

In some embodiments, the said parenteral infusion composition of cabazitaxel does not comprise a lipid (e.g. soybean oil). In some embodiments, the first liquid composition does not comprise a lipid (e.g. soybean oil). In some embodiments, the second aqueous composition does not comprise a lipid (e.g. soybean oil). In some embodiments, the lipid is soybean oil.

In some embodiments, the first liquid composition does not comprise a surfactant. In some embodiments, the second aqueous composition does not comprise a surfactant.

In some embodiments, the said parenteral infusion composition of cabazitaxel is prepared less than 24 hours prior to being infused or administered to patients. In some embodiments, the said parenteral infusion composition of cabazitaxel is prepared less than 8 hours prior to being infused or administered to patients. In some embodiments, the said parenteral infusion composition of cabazitaxel is prepared less than 6 hours prior to being infused or administered to patients. In some embodiments, the said parenteral infusion composition of cabazitaxel is prepared less than 4 hours prior to being infused or administered to patients. In some embodiments, the said parenteral infusion composition of cabazitaxel is prepared less than 1 hour prior to being infused or administered to patients.

In some embodiments, the concentration of cabazitaxel in the parenteral infusion composition is from about 0.01 mg/ml to about 0.5 mg/ml. In some embodiments, the concentration of cabazitaxel in the parenteral infusion composition is from about 0.02 mg/ml to about 0.25 mg/ml. In some embodiments, the concentration of cabazitaxel in the parenteral infusion composition is from about 0.03 mg/ml to about 0.2 mg/ml. In some embodiments, the concentration of cabazitaxel in the parenteral infusion composition is from about 0.03 mg/ml to about 0.15 mg/ml. In some embodiments, the concentration of cabazitaxel in the parenteral infusion composition is from about 0.05 mg/ml to about 0.15 mg/ml. In some embodiments, the weight ratio of human serum albumin to cabazitaxel is from about 10:1 to about 500:1. In some embodiments, the weight ratio of human serum albumin to cabazitaxel is from about 20:1 to about 200:1. In some embodiments, the weight ratio of human serum albumin to cabazitaxel is from about 50:1 to about 150:1. In some embodiments, the weight ratio of human serum albumin to cabazitaxel is about 120:1, about 100:1, about 80:1, about 60:1, or about 50:1.

In some embodiments, the first liquid composition comprises cabazitaxel, ethanol, and other organic solvents (e.g. propylene glycol, polyethylene glycol, hereinafter “PEG”, etc.). The PEG preferably has a molecular weight of about 300, i.e. PEG 300, or about 400, i.e. PEG 400. Other molecular weight PEG's known to those of ordinary skill can be included if desired in alternative embodiments. In some embodiments, the first liquid composition comprises cabazitaxel, ethanol, and propylene glycol. In some embodiments, the first liquid composition comprises cabazitaxel, ethanol, and PEG. In some embodiments, the first liquid composition comprises cabazitaxel, ethanol, and PEG 300. In some embodiments, the first liquid composition comprises cabazitaxel, ethanol, and PEG400. In some embodiments, the first liquid composition further comprises an acid. In some embodiments, the first liquid composition comprises cabazitaxel, ethanol, and an organic acid. The most preferred acid for use in accordance with the present invention is citric acid. In some embodiments, the first liquid composition comprises cabazitaxel, ethanol, and citric acid. In some embodiments, the first liquid composition comprises cabazitaxel, ethanol, propylene glycol, and citric acid. In some embodiments, the first liquid composition comprises cabazitaxel, ethanol, and citric acid. In some embodiments, the first liquid composition comprises cabazitaxel, ethanol, PEG (e.g. PEG 300, PEG 400), and citric acid. In some embodiments, the weight ratio of cabazitaxel and citric acid in the first liquid composition is from about 5000:1 to about 1:1. In some embodiments, the weight ratio of cabazitaxel and citric acid in the first liquid composition is from about 2000:1 to about 1:1. In some embodiments, the weight ratio of cabazitaxel and citric acid in the first liquid composition is from about 1000:1 to about 1:1. In some embodiments, the weight ratio of cabazitaxel and citric acid in the first liquid composition is from about 500:1 to about 1:1. In some embodiments, the weight ratio of cabazitaxel and citric acid in the first liquid composition is from about 5000:1 to about 10:1. In some embodiments, the weight ratio of cabazitaxel and citric acid in the first liquid composition is from about 2000:1 to about 10:1. In some embodiments, the weight ratio of cabazitaxel and citric acid in the first liquid composition is from about 1000:1 to about 10:1. In some embodiments, the weight ratio of cabazitaxel and citric acid in the first liquid composition is from about 500:1 to about 10:1. In some embodiments, the weight ratio of cabazitaxel and citric acid in the first liquid composition is from about 700:1 to about 200:1.

In some embodiments, the first liquid composition is prepared by dissolving cabazitaxel in a solvent comprising ethanol. In some embodiments, the first liquid composition is prepared by dissolving cabazitaxel and citric acid in a solvent comprising ethanol. In some embodiments, the first liquid composition is prepared by dissolving cabazitaxel and citric acid in a solvent comprising ethanol and propylene glycol. In some embodiments, the first liquid composition is prepared by dissolving cabazitaxel and citric acid in a solvent comprising ethanol and PEG (e.g. PEG 300, PEG 400).

In some embodiments, the second aqueous composition is prepared by adding a human serum albumin solution into a parenterally acceptable vehicle. In some embodiments, the second aqueous composition is prepared by adding a human serum albumin solution into an infusion bag or bottle which contains a parenterally acceptable vehicle. In some embodiments, the second aqueous composition is prepared by adding a human serum albumin solution into a normal saline solution or dextrose solution. In some embodiments, the second aqueous composition is prepared by adding a human serum albumin solution into an infusion bag or bottle which contains a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a 20% solution of human serum albumin for infusion (w/v) into a normal saline solution or dextrose solution. In some embodiments, the second aqueous composition is prepared by adding a 25% solution of human serum albumin for infusion (w/v) into a normal saline solution or dextrose solution. In some embodiments, the second aqueous composition is prepared by adding a 5% solution of human serum albumin for infusion (w/v) into a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a 20% solution of human serum albumin for infusion (w/v) into an infusion bag or bottle which contains a normal saline solution or dextrose solution. In some embodiments, the second aqueous composition is prepared by adding a 25% solution of human serum albumin for infusion (w/v) into an infusion bag or bottle which contains a normal saline solution or dextrose solution. In some embodiments, the second aqueous composition is prepared by adding a 5% solution of human serum albumin for infusion (w/v) into an infusion bag or bottle which contains a normal saline solution or dextrose solution. In some embodiments, the second aqueous composition is an aqueous solution comprising human serum albumin. In some embodiments, the concentration of human serum albumin in the second aqueous composition is from about 0.1% to about 20% (w/v). In some embodiments, the concentration of human serum albumin in the second aqueous composition is from about 0.1% to about 10% (w/v). In some embodiments, the concentration of human serum albumin in the second aqueous composition is from 0.2% to 5% (w/v). In some embodiments, the concentration of human serum albumin in the second aqueous composition is from 0.3% to 2% (w/v). In some embodiments, the concentration of human serum albumin in the second aqueous composition is from 0.3% to 1.5% (w/v). In some embodiments, the second aqueous composition comprises from about 0.1 g to about 20 g of human serum albumin. In some embodiments, the second aqueous composition comprises from about 0.5 g to about 10 g of human serum albumin.

The injection of the first liquid composition into the infusion bag or bottle containing the second aqueous composition is rapid. In some embodiments, the injection time is no more than 60 seconds. In some embodiments, the injection time is no more than 30 seconds. In some embodiments, the injection time is no more than 15 seconds. In some embodiments, the injection time is no more than 10 seconds. In some embodiments, the injection time is no more than 5 seconds.

In the process of injecting the first liquid composition into the infusion bag or bottle containing the second aqueous composition, mixing or agitation is not need. After the injection of the first liquid composition into the infusion bag or bottle is completed, the first liquid composition and second aqueous composition are mixed well (e.g., the bag containing the composition is gently inverted by hand) to obtain a transparent infusion solution free of precipitates. In some embodiments, the infusion bag or bottle containing the second aqueous composition is held still in the injection process. In some embodiments, the first liquid composition is injected underneath the liquid surface of the second aqueous composition in the injection process. In some embodiments, after the injection is completed, the infusion bag or bottle is gently inverted repeatedly to mix well of the first liquid composition and second aqueous composition. In some embodiments, after the injection is completed, the infusion bag or bottle is gently inverted until a transparent solution free of precipitates is obtained. In some embodiments, after the injection is completed, the infusion bag or bottle is gently inverted for from about 5 seconds to about 10 minutes. In some embodiments, after the injection is completed, the infusion bag or bottle is gently inverted for from about 10 seconds to about 5 minutes. In some embodiments, after the injection is completed, the infusion bag or bottle is gently inverted for from about 0.5 minute to about 3 minutes. In some embodiments, after the injection is completed, the first liquid composition and second aqueous composition are mixed well until a transparent infusion solution free of precipitates is obtained.

After the first liquid composition and second aqueous composition are mixed well followed the injection of the first liquid composition into the infusion bag or bottle comprising the second aqueous composition, a transparent infusion solution free of precipitates comprising human serum albumin, cabazitaxel, and ethanol in a parenterally acceptable vehicle is obtained. In some embodiments, the transparent infusion solution further comprises citric acid. In some embodiments, a parenterally acceptable vehicle is a normal saline solution or dextrose solution. In some embodiments, the transparent infusion solution stays transparent free of precipitates for at least 1 hour. In some embodiments, the transparent infusion solution stays transparent free of precipitates for at least 2 hours. In some embodiments, the transparent infusion solution stays transparent free of precipitates for at least 3 hours. In some embodiments, the transparent infusion solution stays transparent free of precipitates for at least 4 hours. In some embodiments, the transparent infusion solution stays transparent free of precipitates for at least 6 hours. In some embodiments, the transparent infusion solution stays transparent free of precipitates for at least 8 hours. In some embodiments, the transparent infusion solution stays transparent free of precipitates for at least 1 hour, 2 hours, 3 hours, 4 hours, or 6 hours, when the transparent solution is kept at about 20-25° C. In some embodiments, the transparent solution stays transparent free of precipitates for at least 2 hours, 4 hours, 6 hours, 8 hours, 12 hours, or 24 hours, when the transparent solution is kept at about 2-8° C.

Also, provided herein is a kit for the preparation of a parenteral infusion solution comprising: a first container containing a composition comprising cabazitaxel and a second container containing a composition comprising human serum albumin. In some embodiments, the kit comprises a first container containing a liquid composition comprising cabazitaxel and a second container containing a liquid composition comprising human serum albumin.

In some embodiments, the first container does not comprise a lipid (e.g. soybean oil). In some embodiments, the second container does not comprise a lipid (e.g. soybean oil). In some embodiments, the lipid is soybean oil. In some embodiments, the first container does not comprise a surfactant. In some embodiments, the second container does not comprise a surfactant.

In some embodiments, the first container contains a liquid composition comprising cabazitaxel and ethanol. In some embodiments, the first container contains a liquid composition comprising cabazitaxel, an acid, and ethanol. In some embodiments, the first container contains a liquid composition comprising cabazitaxel, an organic acid, and ethanol. In some embodiments, the first container contains a liquid composition comprising cabazitaxel, ethanol, and other organic solvents (e.g., alcohols such as propylene glycol, polyethylene glycol 300, etc.). In some embodiments, the first container contains a liquid composition comprising cabazitaxel, ethanol, and propylene glycol. In some embodiments, the first container contains a liquid composition comprising cabazitaxel, ethanol, and polyethylene glycol 300. In some embodiments, the first container contains a liquid composition comprising cabazitaxel, ethanol, and polyethylene glycol 400. In some embodiments, the first container contains a liquid composition comprising cabazitaxel and citric acid. In some embodiments, the first container contains a liquid composition comprising cabazitaxel, citric acid, and ethanol. In some embodiments, the first container contains a liquid composition comprising cabazitaxel, citric acid, ethanol, and propylene glycol. In some embodiments, the weight ratio of cabazitaxel and citric acid in the first container is from about 5000:1 to about 1:1. In some embodiments, the weight ratio of cabazitaxel and citric acid in the first container is from about 2000:1 to about 1:1. In some embodiments, the weight ratio of cabazitaxel and citric acid in the first container is from about 1000:1 to about 1:1. In some embodiments, the weight ratio of cabazitaxel and citric acid in the first container is from about 500:1 to about 1:1. In some embodiments, the weight ratio of cabazitaxel and citric acid in the first container is from about 5000:1 to about 10:1. In some embodiments, the weight ratio of cabazitaxel and citric acid in the first container is from about 2000:1 to about 10:1. In some embodiments, the weight ratio of cabazitaxel and citric acid in the first container is from about 1000:1 to about 10:1. In some embodiments, the weight ratio of cabazitaxel and citric acid in the first container is from about 500:1 to about 10:1. In some embodiments, the weight ratio of cabazitaxel and citric acid in the first container is from about 700:1 to about 200:1.

In some embodiments, the amount of cabazitaxel contained in the first container is from about 5 mg to about 100 mg of cabazitaxel. In some embodiments, the amount of cabazitaxel contained in the first container is from about 5 mg to about 80 mg of cabazitaxel. In some embodiments, the amount of cabazitaxel contained in the first container is from about 10 mg to about 60 mg of cabazitaxel. In some embodiments, the amount of cabazitaxel contained in the first container is about 10 mg, about 20 mg, about 30 mg, about 40 mg, about 50 mg, or about 60 mg of cabazitaxel. In some embodiments, the concentration of cabazitaxel in the first container is from about 1 mg/ml to about 50 mg/ml. In some embodiments, the concentration of cabazitaxel in the first container is from about 5 mg/ml to about 40 mg/ml. In some embodiments, the concentration of cabazitaxel in the first container is from about 5 mg/ml to about 20 mg/ml. In some embodiments, the concentration of cabazitaxel in the first container is about 10 mg/ml or 5 mg/ml.

In some embodiments, the first container contains a solid composition comprising cabazitaxel. In some embodiments, the first container contains a solid composition comprising cabazitaxel and an organic acid. In some embodiments, the first container contains a solid composition comprising cabazitaxel and citric acid.

In some embodiments, the second container contains a liquid composition comprising human serum albumin. In some embodiments, the second container contains a liquid composition comprising human serum albumin with the concentration of human serum albumin in the composition from about 1% to about 25% (w/v). In some embodiments, the second container contains a human serum albumin solution. In some embodiments, the second container contains a human serum albumin solution with the concentration of human serum albumin in the solution from about 1% to about 25% (w/v). In some embodiments, the second container contains a human serum albumin solution with the concentration of human serum albumin in the solution from about 5% to about 25% (w/v). In some embodiments, the second container contains a solution of human serum albumin for infusion. In some embodiments, the second container contains a 20% solution of human serum albumin for infusion (w/v). In some embodiments, the second container contains a 25% solution of human serum albumin for infusion (w/v). In some embodiments, the second container contains a 5% solution of human serum albumin for infusion (w/v). In some embodiments, the second container contains a liquid composition comprising from about 1 g to about 50 g of human serum albumin. In some embodiments, the second container contains a liquid composition comprising from about 1 g to about 20 g of human serum albumin. In some embodiments, the second container contains a liquid composition comprising from about 1 g to about 10 g of human serum albumin. In some embodiments, the second container contains a liquid composition comprising from about 10 g of human serum albumin.

In some embodiments, the kit further comprises instructions to add the composition comprising cabazitaxel to the composition comprising human serum albumin.

Also, provided herein is a liquid composition comprising cabazitaxel and ethanol, wherein the composition does not contain a surfactant (e.g. Polysorbate 80), and wherein the said liquid composition is mixed with a second aqueous composition comprising human serum albumin to form an infusion solution comprising cabazitaxel prior to being infused or administered to patients. In some embodiments, the two compositions are mixed in less than or equal to about 24 hours prior to being infused or administered to patients. In some embodiments, the two compositions are mixed in less than or equal to about 8 hours prior to being infused or administered to patients. In some embodiments, the two compositions are mixed in less than or equal to about 6 hours prior to being infused or administered to patients. In some embodiments, the two compositions are mixed in less than or equal to about 2 hours prior to being infused or administered to patients. In some embodiments, the two compositions are mixed in less than or equal to about 1 hour prior to being infused or administered to patients. In some embodiments, the mixing is done in an infusion bag or infusion bottle. In some embodiments, the second aqueous composition comprises a parenterally acceptable vehicle. In some embodiments, the second aqueous composition is contained in an infusion bag or infusion bottle. In some embodiments, the liquid composition is injected into an infusion bag or infusion bottle which contains the second aqueous composition comprising human serum albumin and a parenterally acceptable vehicle. In some embodiments, the liquid composition further comprises citric acid.

### BRIEF DESCRIPTION OF THE DRAWINGS

FIG. 1 is a line plot showing mean plasma concentration-time profile of cabazitaxel in rats administered composition solution following a single intravenous infusion.

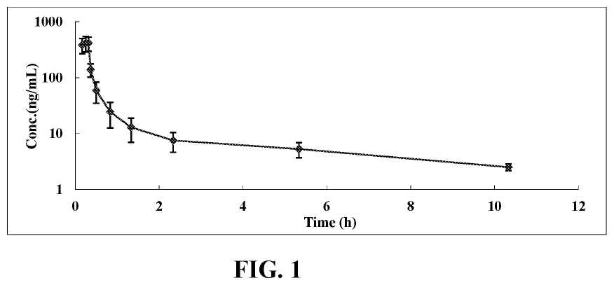

### DETAILED DESCRIPTION

Provided herein is a pharmaceutical formulation of cabazitaxel comprising two compositions that are mixed prior to being infused or administered to patients, that comprises: (a) a first liquid composition comprising cabazitaxel and ethanol, wherein the cabazitaxel is dissolved in a solvent comprising ethanol, and (b) a second aqueous composition comprising human serum albumin and a parenterally acceptable vehicle, and wherein the said pharmaceutical formulation does not contain a surfactant (e.g. Polysorbate 80). In some embodiments, the two compositions are mixed in less than or equal to about 24 hours prior to being infused or administered to patients. In some embodiments, the two compositions are mixed in less than or equal to about 8 hours prior to being infused or administered to patients. In some embodiments, the two compositions are mixed in less than or equal to about 6 hours prior to being infused or administered to patients. In some embodiments, the two compositions are mixed in less than or equal to about 2 hours prior to being infused or administered to patients. In some embodiments, the two compositions are mixed in less than or equal to about 1 hour prior to being infused or administered to patients. In some embodiments, the mixing of the two compositions is done in an infusion bag or infusion bottle. In some embodiments, the second aqueous composition is contained in an infusion bag or bottle (e.g. 500 ml or 250 ml size). In some embodiments, the second aqueous composition is prepared by adding a clinically-used solution of human serum albumin for infusion into an infusion bag or bottle which contains a parenterally acceptable vehicle. In some embodiments, the first liquid composition is injected into an infusion bag or bottle which contains the second aqueous composition. In some embodiments, the desired amount of the first liquid composition (e.g. from about 0.5 ml to about 15 ml) in a syringe is injected into an infusion bag or bottle which contains the desired amount of the second aqueous composition (e.g. from about 100 ml to about 500 ml) to prepare an infusion solution prior to being infused or administered to patients. In some embodiments, mixing (e.g., manual mixing such as with a stirrer) or agitation is not need in the process of injecting the first liquid composition into an infusion bag or bottle which contains the second aqueous composition. In some embodiments, both the first liquid composition and the second aqueous composition are sterile solutions. In some embodiments, the first liquid composition is a sterile solution. In some embodiments, the second aqueous composition is a sterile solution. In some embodiments, the mixing of the first liquid composition and the second aqueous composition is done in a hospital or clinic prior to being infused or administered to patients.

In some embodiments, the said pharmaceutical formulation does not comprise a lipid (e.g. soybean oil). In some embodiments, the first liquid composition does not comprise a lipid (e.g. soybean oil). In some embodiments, the second aqueous composition does not comprise a lipid (e.g. soybean oil). In some embodiments, the lipid is soybean oil.

In some embodiments, the said pharmaceutical formulation does not comprise a surfactant. In some embodiments, the first liquid composition does not comprise a surfactant. In some embodiments, the second aqueous composition does not comprise a surfactant.

In some embodiments, the concentration of cabazitaxel in the infusion solution after mixing of the first liquid composition and the second aqueous composition is from about 0.01 mg/ml to about 0.5 mg/ml. In some embodiments, the concentration of cabazitaxel in the infusion solution after mixing of the first liquid composition and the second aqueous composition is from about 0.02 mg/ml to about 0.2 mg/ml. In some embodiments, the concentration of cabazitaxel in the infusion solution after mixing of the first liquid composition and the second aqueous composition is from about 0.03 mg/ml to about 0.15 mg/ml. In some embodiments, the concentration of cabazitaxel in the infusion solution after mixing of the first liquid composition and the second aqueous composition is from about 0.03 mg/ml to about 0.1 mg/ml. In some embodiments, the concentration of cabazitaxel in the infusion solution after mixing of the first liquid composition and the second aqueous composition is about 0.08 mg/ml or about 0.1 mg/ml.

In some embodiments, at least 10% cabazitaxel in the infusion solution after mixing of the first liquid composition and the second aqueous composition is Free (unbound in solution) cabazitaxel. In some embodiments, at least 20% cabazitaxel in the infusion solution after mixing of the first liquid composition and the second aqueous composition is Free (unbound in solution) cabazitaxel. In some embodiments, at least 30% cabazitaxel in the infusion solution after mixing of the first liquid composition and the second aqueous composition is Free (unbound in solution) cabazitaxel. In some embodiments, at least 40% cabazitaxel in the infusion solution after mixing of the first liquid composition and the second aqueous composition is Free (unbound in solution) cabazitaxel. In some embodiments, at least 50% cabazitaxel in the infusion solution after mixing of the first liquid composition and the second aqueous composition is Free (unbound in solution) cabazitaxel. In some embodiments, from about 10% to about 90% of cabazitaxel in the infusion solution after mixing of the first liquid composition and the second aqueous composition is Free (unbound in solution) cabazitaxel. In some embodiments, from about 20% to about 80% of cabazitaxel in the infusion solution after mixing of the first liquid composition and the second aqueous composition is Free (unbound in solution) cabazitaxel. In some embodiments, from about 30% to about 70% of cabazitaxel in the infusion solution after mixing of the first liquid composition and the second aqueous composition is Free (unbound in solution) cabazitaxel. Fraction of free (unbound in solution) cabazitaxel is measured by ultrafiltration through a 30-kDa membrane. The experiments of free (unbound in solution) cabazitaxel measurement are described in the example section.

In some embodiments, the pharmaceutical formulation of cabazitaxel comprising two compositions that are mixed prior to being infused or administered to patients, that comprises: (a) a first liquid composition comprising from about 5 mg to about 60 mg of cabazitaxel and from about 0.5 ml to about 15 ml of ethanol, and (b) a second aqueous composition comprising from about 0.5 g to about 20 g of human serum albumin in a parenterally acceptable vehicle. In some embodiments, the pharmaceutical formulation of cabazitaxel comprising two compositions that are mixed prior to being infused or administered to patients, that comprises: (a) a first liquid composition comprising citric acid, from about 5 mg to about 60 mg of cabazitaxel, and ethanol, and (b) a second aqueous composition comprising from about 0.5 g to about 20 g of human serum albumin in a parenterally acceptable vehicle. In some embodiments, the pharmaceutical formulation of cabazitaxel comprising two compositions that are mixed prior to being infused or administered to patients, that comprises: (a) a first liquid composition comprising from about 10 mg to about 50 mg of cabazitaxel, citric acid, and ethanol, and (b) a second aqueous composition comprising from about 1 g to about 10 g of human serum albumin in a parenterally acceptable vehicle. In some embodiments, the first liquid composition comprises from about 15 mg to about 40 mg of cabazitaxel, citric acid, and ethanol. In some embodiments, the second aqueous composition comprises from about 1.5 g to about 10 g of human serum albumin. In some embodiments, the second aqueous composition comprises from about 2 g to about 5 g of human serum albumin. In some embodiments, the pharmaceutical formulation does not comprise a lipid (e.g. soybean oil). In some embodiments, the pharmaceutical formulation does not comprise a surfactant.

In some embodiments, the first liquid composition is contained in a syringe. In some embodiments, the first liquid composition comprises cabazitaxel, ethanol, and other organic solvents (e.g. propylene glycol, polyethylene glycol, hereinafter “PEG”, etc.). The PEG preferably has a molecular weight of about 300, i.e. PEG 300, or about 400, i.e. PEG 400. Other molecular weight PEG's known to those of ordinary skill can be included if desired in alternative embodiments. In some embodiments, the first liquid composition comprises cabazitaxel, ethanol, and propylene glycol. In some embodiments, the first liquid composition comprises cabazitaxel, ethanol, and PEG. In some embodiments, the first liquid composition comprises cabazitaxel, ethanol, and PEG 300. In some embodiments, the first liquid composition comprises cabazitaxel, ethanol, and PEG400. In some embodiments, the first liquid composition further comprises an acid. In some embodiments, the first liquid composition further comprises a citric acid. In some embodiments, the first liquid composition comprises cabazitaxel, ethanol, and an organic acid. The most preferred acid for use in accordance with the present invention is citric acid. In some embodiments, the first liquid composition comprises cabazitaxel, ethanol, and citric acid. In some embodiments, the first liquid composition comprises cabazitaxel, ethanol, propylene glycol, and citric acid. In some embodiments, the first liquid composition comprises cabazitaxel, ethanol, and citric acid. In some embodiments, the first liquid composition comprises cabazitaxel, ethanol, PEG (e.g., PEG 300, PEG 400), and citric acid. In some embodiments, the weight ratio of cabazitaxel and citric acid in the first liquid composition is from about 5000:1 to about 1:1. In some embodiments, the weight ratio of cabazitaxel and citric acid in the first liquid composition is from about 2000:1 to about 1:1. In some embodiments, the weight ratio of cabazitaxel and citric acid in the first liquid composition is from about 1000:1 to about 1:1. In some embodiments, the weight ratio of cabazitaxel and citric acid in the first liquid composition is from about 500:1 to about 1:1. In some embodiments, the weight ratio of cabazitaxel and citric acid in the first liquid composition is from about 5000:1 to about 5:1. In some embodiments, the weight ratio of cabazitaxel and citric acid in the first liquid composition is from about 2000:1 to about 5:1. In some embodiments, the weight ratio of cabazitaxel and citric acid in the first liquid composition is from about 1000:1 to about 5:1. In some embodiments, the weight ratio of cabazitaxel and citric acid in the first liquid composition is from about 500:1 to about 5:1. In some embodiments, the weight ratio of cabazitaxel and citric acid in the first liquid composition is from about 5000:1 to about 10:1. In some embodiments, the weight ratio of cabazitaxel and citric acid in the first liquid composition is from about 2000:1 to about 10:1. In some embodiments, the weight ratio of cabazitaxel and citric acid in the first liquid composition is from about 1000:1 to about 10:1. In some embodiments, the weight ratio of cabazitaxel and citric acid in the first liquid composition is from about 500:1 to about 10:1. In some embodiments, the weight ratio of cabazitaxel and citric acid in the first liquid composition is from about 2000:1 to about 100:1. In some embodiments, the weight ratio of cabazitaxel and citric acid in the first liquid composition is from about 1000:1 to about 100:1. In some embodiments, the weight ratio of cabazitaxel and citric acid in the first liquid composition is from about 1000:1 to about 50:1. In some embodiments, the weight ratio of cabazitaxel and citric acid in the first liquid composition is from about 700:1 to about 100:1. In some embodiments, the weight ratio of cabazitaxel and citric acid in the first liquid composition is from about 700:1 to about 20:1. In some embodiments, the weight ratio of cabazitaxel and citric acid in the first liquid composition is from about 700:1 to about 200:1.

In some embodiments, the first liquid composition comprises cabazitaxel and a solvent including ethanol, wherein cabazitaxel is dissolved in the solvent. In some embodiments, the first liquid composition comprises cabazitaxel, an acid, and a solvent including ethanol, wherein cabazitaxel and the acid are dissolved in the solvent. In some embodiments, the first liquid composition comprises cabazitaxel, an organic acid, and a solvent including ethanol, wherein cabazitaxel and the organic acid are dissolved in the solvent. In some embodiments, the first liquid composition comprises cabazitaxel, citric acid, and a solvent including ethanol, wherein cabazitaxel and citric acid are dissolved in the solvent. In some embodiments, the first liquid composition comprises cabazitaxel and ethanol, wherein cabazitaxel is dissolved in ethanol. In some embodiments, the first liquid composition comprises cabazitaxel, citric acid, and ethanol, wherein cabazitaxel and citric acid are dissolved in ethanol. In some embodiments, the first liquid composition comprises cabazitaxel and dehydrated ethanol, wherein cabazitaxel is dissolved in dehydrated ethanol. In some embodiments, the first liquid composition comprises cabazitaxel, citric acid, and dehydrated ethanol, wherein cabazitaxel and citric acid are dissolved in dehydrated ethanol. In some embodiments, the first liquid composition does not comprise a lipid (e.g. soybean oil) or a surfactant.

In some embodiments, the first liquid composition is prepared by dissolving cabazitaxel in a solvent comprising ethanol. In some embodiments, the first liquid composition is prepared by dissolving cabazitaxel and citric acid in a solvent comprising ethanol. In some embodiments, the first liquid composition is prepared by dissolving cabazitaxel and citric acid in ethanol. In some embodiments, the first liquid composition is prepared by dissolving cabazitaxel and citric acid in a solvent comprising ethanol and propylene glycol. In some embodiments, the first liquid composition is prepared by dissolving cabazitaxel and citric acid in a solvent comprising ethanol and PEG (e.g., PEG 300, PEG 400). In some embodiments, the volume of the first liquid composition is from about 0.5 ml to about 30 ml. In some embodiments, the volume of first liquid composition is from about 1 ml to about 20 ml. In some embodiments, the volume of first liquid composition is from about 1 ml to about 10 ml. In some embodiments, the volume of first liquid composition is from about 1 ml to about 5 ml.

In some embodiments, the second aqueous composition is prepared by adding a human serum albumin solution into a parenterally acceptable vehicle. In some embodiments, the second aqueous composition is prepared by adding a human serum albumin solution into an infusion bag or bottle which contains a parenterally acceptable vehicle. In some embodiments, the second aqueous composition is prepared by adding a human serum albumin solution into a normal saline solution or dextrose solution. In some embodiments, the second aqueous composition is prepared by adding a human serum albumin solution into an infusion bag or bottle which contains a normal saline solution or dextrose solution. In some embodiments, the second aqueous composition is prepared by adding a solution of human serum albumin for infusion into a parenterally acceptable vehicle. In some embodiments, the second aqueous composition is prepared by adding a solution of human serum albumin for infusion into a normal saline solution or dextrose solution. In some embodiments, the second aqueous composition is prepared by adding a 20% solution of human serum albumin for infusion (w/v) into a normal saline solution or dextrose solution. In some embodiments, the second aqueous composition is prepared by adding a 25% solution of human serum albumin for infusion (w/v) into a normal saline solution or dextrose solution. In some embodiments, the second aqueous composition is prepared by adding a 5% solution of human serum albumin for infusion (w/v) into a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a solution of clinically used human serum albumin for infusion (e.g. 20%, 25%, 5%, w/v) into an infusion bag or bottle which contains a normal saline solution or dextrose solution. In some embodiments, the second aqueous composition is prepared by adding a solution of clinically used human serum albumin for infusion containing from about 1 g to about 10 g of human serum albumin into an infusion bag or bottle which contains a normal saline solution or dextrose solution. In some embodiments, the second aqueous composition is prepared by adding a 20% solution of human serum albumin for infusion (w/v) into an infusion bag or bottle which contains a normal saline solution or dextrose solution. In some embodiments, the second aqueous composition is prepared by adding a 25% solution of human serum albumin for infusion (w/v) into an infusion bag or bottle which contains a normal saline solution or dextrose solution. In some embodiments, the second aqueous composition is prepared by adding a 5% solution of human serum albumin for infusion (w/v) into an infusion bag or bottle which contains a normal saline solution or dextrose solution. In some embodiments, the second aqueous composition is an aqueous solution comprising human serum albumin. In some embodiments, the concentration of human serum albumin in the second aqueous composition is from about 0.0010% to about 20% (w/v). In some embodiments, the concentration of human serum albumin in the second aqueous composition is from about 0.01% to about 20% (w/v). In some embodiments, the concentration of human serum albumin in the second aqueous composition is from about 0.1% to about 20% (w/v). In some embodiments, the concentration of human serum albumin in the second aqueous composition is from about 0.2% to about 15% (w/v). In some embodiments, the concentration of human serum albumin in the second aqueous composition is from about 0.2% to about 10% (w/v). In some embodiments, the concentration of human serum albumin in the second aqueous composition is from 0.2% to 5% (w/v). In some embodiments, the concentration of human serum albumin in the second aqueous composition is from about 0.5% to about 2.5% (w/v). In some embodiments, the concentration of human serum albumin in the second aqueous composition is from 0.5% to 1.5% (w/v). In some embodiments, the concentration of human serum albumin in the second aqueous composition is from 0.2% to 1.5% (w/v). In some embodiments, the second aqueous composition comprises from about 0.05 g to about 20 g of human serum albumin. In some embodiments, the second aqueous composition comprises from about 0.1 g to about 20 g of human serum albumin. In some embodiments, the second aqueous composition comprises from about 1 g to about 20 g of human serum albumin. In some embodiments, the second aqueous composition comprises from about 1 g to about 15 g of human serum albumin. In some embodiments, the second aqueous composition comprises from about 1.5 g to about 10 g of human serum albumin. In some embodiments, the second aqueous composition comprises from about 1 g to about 5 g of human serum albumin. In some embodiments, the volume of the second aqueous composition is from about 100 ml to about 1 L. In some embodiments, the volume of the second aqueous composition is from about 100 ml to about 600 ml. In some embodiments, the volume of the second aqueous composition is from about 150 ml to about 500 ml. In some embodiments, the volume of the second aqueous composition is from about 150 ml to about 450 ml. In some embodiments, the volume of the second aqueous composition is from about 250 ml to about 500 ml. In some embodiments, the volume of the second aqueous composition is from about 250 ml to about 450 ml.

As used herein the term “cabazitaxel” refers to a compound that has the CAS No. 183133-96-2 and the following chemical structure:

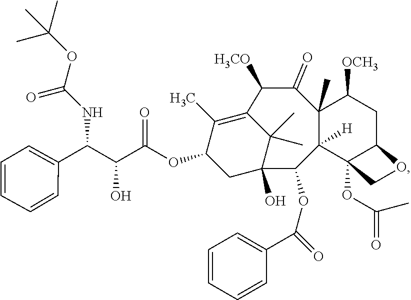

or a pharmaceutically acceptable salt thereof.

Cabazitaxel is lipophilic, practically insoluble in water and soluble in alcohol.

Further, cabazitaxel is a microtubule inhibitor indicated in combination with prednisone for treatment of patients with hormone-refractory metastatic prostate cancer previously treated with a docetaxel-containing treatment regimen.

In some embodiments, the term “cabazitaxel” refers to a pharmaceutically acceptable salt of cabazitaxel.

As used herein, the term “pharmaceutically acceptable salts” refers to salts that retain the desired biological activity of a compound (e.g., cabazitaxel, arginine) and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), napthalene-2-sulfonate, ethanedisulfonate, hydrogen bisulfide, bitartrate, gluconate, glucuronate, para-bromophenylsulfonate, carbonate, pyrosulfate, sulfite, bisulfite, monohydrogen phosphate, dihydrogen phosphate, metaphosphate, pyrophosphate, chloride, bromide, iodide, decanoate, caprylate, caprate, propiolate, suberate, sebacate, butyne-1,4-dioate, hexyne-1,6-dioate, terephthalate, sulfonate, xylenesulfonate, phenylpropionate, phenylbutyrate, β-hydroxybutyrate, glycolate, propanesulfonate, naphthalene-1-sulfonate, naphthalene-2-sulfonate and 2,5-dihydroxybenzoate. Suitable bases include pharmaceutically acceptable inorganic bases and pharmaceutically acceptable organic bases. Representative pharmaceutically acceptable base addition salts include hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the cabazitaxel can be a cabazitaxel with one equivalent of the acetone solvate. In some embodiments, cabazitaxel, or a salt thereof, may be crystalline or amorphous. In some embodiments, cabazitaxel, or a salt thereof, may be in a form of a hydrate. In some embodiments, the cabazitaxel can be any one of cabazitaxel solvates, hydrates, and/or crystal forms disclosed, for example, in US application publication No. 20150315164, US application publication No. 20160257663, US application publication No. 20160340327, US application publication No. 20160244420, US application publication No. 20150141673, U.S. Pat. Nos. 9,012,665, 9,353,076, 9,394,266, 9,309,210, 9,199,953, 8,735,611, 8,735,611, 8,901,322, PCT publication No. WO2014115168, PCT publication No. WO2015087228, PCT publication No. WO2014067207, PCT publication No. WO2014128728 or PCT publication No. WO2015058960, the disclosures of each of the above are incorporated herein by reference in their entirety.

As used herein, the term “human serum albumin” refers to native and recombinant human serum albumin. In some embodiments, the human serum albumin is a native human serum albumin. In some embodiments, the human serum albumin is a recombinant human serum albumin.

In some embodiments, “human serum albumin” refers to human serum albumin solution. In some embodiments, “human serum albumin” refers to commercially available solutions of human serum albumin for infusion (e.g., 20%, 5%, 25% (w/v)). Suitable human serum albumin solution include, but are not limited to, commercially available solutions of human serum albumin for infusion. The commercially available solutions of human serum albumin for infusion comprise pharmaceutically acceptable excipient(s) such as sodium N-acetyltryptophan, sodium caprylate, sodium chloride, sodium bicarbonate, sodium hydroxide, or acetic acid, and the like or mixtures thereof. In some embodiments, human serum albumin solution can be prepared by diluting commercially available solutions of human serum albumin for infusion with a parenterally acceptable vehicle.

Alternatively, human serum albumin solution can be prepared by mixing human serum albumin powder in water along with other pharmaceutically acceptable excipient(s) as available in the commercially available albumin products.

In some embodiments, the human serum albumin solution is a commercially available solution of human serum albumin USP for infusion. In some embodiments, the human serum albumin solution for infusion. is a commercially available solution of human serum albumin USP for infusion. In some embodiments, the human serum albumin solution comprises a commercially available solution of human serum albumin USP for infusion. In some embodiments, a commercially available solution of human serum albumin USP for infusion is used as the source of the human serum albumin solution. In some embodiments, the solution of human serum albumin for infusion is 5% solution of human serum albumin USP (w/v). In some embodiments, the solution of human serum albumin for infusion is 20% solution of human serum albumin USP (w/v). In some embodiments, the solution of human serum albumin for infusion is 25% solution of human serum albumin USP (w/v). In some embodiments, the human serum albumin solution is an aqueous solution prepared by diluting a commercially available solution of human serum albumin for infusion.

The term “parenteral” refers to routes selected from subcutaneous (SC), intravenous (IV), intramuscular (IM), intrademal (ID), intraperitoneal (IP) and the like.

Also, provided herein is a transparent parenteral infusion composition of cabazitaxel free of visible particles or precipitates comprising human serum albumin and said cabazitaxel in a concentration of cabazitaxel from about 0.01 mg/ml to about 0.5 mg/ml in a parenterally acceptable vehicle, wherein the said parenteral infusion solution is obtained by injecting a first liquid composition comprising cabazitaxel and ethanol into an infusion bag or bottle containing a second aqueous composition comprising human serum albumin in a parenterally acceptable vehicle, wherein mixing or agitation is not needed in the injection process. In some embodiments, the said infusion solution does not contain a surfactant (e.g. Polysorbate 80). In some embodiments, a parenterally acceptable vehicle is a normal saline solution or a dextrose solution. In some embodiments, the transparent infusion solution further comprises citric acid. In some embodiments, the transparent infusion solution stays transparent without visible particles or precipitates for at least 2 hours at temperature below about 25° C.

Also, provided herein is an infusion composition (e.g., parenteral infusion composition) comprising cabazitaxel, human serum albumin, and ethanol in an infusion bag or bottle. In some embodiments, the infusion composition is an aqueous composition (e.g., aqueous solution). In some embodiments, the infusion composition is substantially free of visible particles or precipitates. In some embodiments, the infusion composition is substantially free from surfactant. In some embodiments, the infusion solution does not contain a surfactant. In some embodiments, the infusion composition does not contain a Polysorbate 80. In some embodiments, concentration of cabazitaxel in the infusion composition is from about 0.05 mg/ml to about 0.2 mg/ml, concentration of ethanol in the infusion composition is from about 0.5 vol. % to about 4 vol. %, and weight ratio of human serum albumin to cabazitaxel in the infusion composition is from about 10:1 to about 200:1.

Also, provided herein is a method of making an infusion composition comprising cabazitaxel, human serum albumin, and ethanol, the method comprising (i) obtaining a first liquid composition comprising cabazitaxel and ethanol; (ii) obtaining a second liquid composition comprising human serum albumin in a parenterally acceptable vehicle in an infusion bag or bottle; and (iii) injecting the first liquid composition into the infusion bag or bottle containing the second liquid composition to obtain the infusion composition comprising cabazitaxel, human serum albumin, and ethanol. In some embodiments, the first liquid composition is contained in a syringe. In some embodiments, mixing or agitation is not needed in the injection process. Also, provided herein is an infusion composition prepared by mixing a first liquid composition containing cabazitaxel and ethanol with a second aqueous composition containing human serum albumin in a parenterally acceptable vehicle (e.g., saline or dextrose solution).

Certain embodiments of the infusion composition, the first liquid composition, the second aqueous composition, and the preparation processes thereof are described herein.

In some embodiments, at least 10% cabazitaxel in the infusion composition is Free (unbound in solution) cabazitaxel. In some embodiments, at least 20% cabazitaxel in the infusion composition is Free (unbound in solution) cabazitaxel. In some embodiments, at least 30% cabazitaxel in the infusion composition is Free (unbound in solution) cabazitaxel. In some embodiments, at least 40% cabazitaxel in the infusion composition is Free (unbound in solution) cabazitaxel. In some embodiments, at least 50% cabazitaxel in the infusion composition is Free (unbound in solution) cabazitaxel. In some embodiments, from about 10% to about 90% of cabazitaxel in the infusion composition is Free (unbound in solution) cabazitaxel. In some embodiments, from about 20% to about 80% of cabazitaxel in the infusion composition is Free (unbound in solution) cabazitaxel. In some embodiments, from about 30% to about 70% of cabazitaxel in the infusion composition is Free (unbound in solution) cabazitaxel. Fraction of free (unbound in solution) cabazitaxel in the infusion composition is measured by ultrafiltration through a 30-kDa membrane. The experiments of free (unbound in solution) cabazitaxel measurement are described in the example section.

In some embodiments, the said parenteral infusion composition of cabazitaxel does not comprise a lipid (e.g. soybean oil). In some embodiments, the first liquid composition does not comprise a lipid (e.g. soybean oil). In some embodiments, the second aqueous composition does not comprise a lipid (e.g. soybean oil). In some embodiments, the lipid is soybean oil.

In some embodiments, the first liquid composition does not comprise a surfactant. In some embodiments, the second aqueous composition does not comprise a surfactant.

In some embodiments, the amount of ethanol in the parenteral infusion composition of cabazitaxel is no more than 5% (v/v). In some embodiments, the amount of ethanol in the parenteral infusion composition of cabazitaxel is no more than 3% (v/v). In some embodiments, the amount of ethanol in the parenteral infusion composition of cabazitaxel is no more than 2% (v/v). In some embodiments, the amount of ethanol in the parenteral infusion composition of cabazitaxel is no more than 1.5% (v/v). In some embodiments, the amount of ethanol in the parenteral infusion composition of cabazitaxel is no more than 1% (v/v). In some embodiments, the amount of ethanol in the parenteral infusion composition of cabazitaxel is no more than 0.8% (v/v). In some embodiments, the amount of ethanol in the parenteral infusion composition of cabazitaxel is no more than 0.7% (v/v). In some embodiments, the amount of ethanol in the parenteral infusion composition of cabazitaxel is no more than 30 ml. In some embodiments, the amount of ethanol in the parenteral infusion composition of cabazitaxel is no more than 15 ml. In some embodiments, the amount of ethanol in the parenteral infusion composition of cabazitaxel is no more than 10 ml. In some embodiments, the amount of ethanol in the parenteral infusion composition of cabazitaxel is no more than 5 ml. In some embodiments, concentration of ethanol in the infusion composition is from about 0.1 vol. % to about 3 vol. %, from about 0.5 vol. % to about 2 vol. %, or from about 0.7 vol. % to about 1.5 vol. %. In some embodiments, concentration of ethanol in the infusion composition is about 0.5 vol. %, about 0.7 vol. %, about 0.8 vol. %, about 1 vol. %, or about 2 vol. %.

In some embodiments, the amount of human serum albumin in the parenteral infusion composition of cabazitaxel is from about 0.1% (w/v) to about 5% (w/v). In some embodiments, the amount of human serum albumin in the parenteral infusion composition of cabazitaxel is from about 0.2% (w/v) to about 5% (w/v).

In some embodiments, the said parenteral infusion composition of cabazitaxel is prepared less than 24 hours prior to being infused or administered to patients. In some embodiments, the said parenteral infusion composition of cabazitaxel is prepared less than 8 hours prior to being infused or administered to patients. In some embodiments, the said parenteral infusion composition of cabazitaxel is prepared less than 6 hours prior to being infused or administered to patients. In some embodiments, the said parenteral infusion composition of cabazitaxel is prepared less than 4 hours prior to being infused or administered to patients. In some embodiments, the said parenteral infusion composition of cabazitaxel is prepared less than 1 hour prior to being infused or administered to patients.

In some embodiments, the pH value of the parenteral infusion composition is from about 4 to about 9.5. In some embodiments, the pH value of the parenteral infusion composition is from about 5 to about 9. In some embodiments, the pH value of the parenteral infusion composition is from about 5 to about 8. In some embodiments, the pH value of the parenteral infusion composition is from about 6 to about 8. In some embodiments, the pH value of the parenteral infusion composition is from about 6.5 to about 7.5. In some embodiments, the pH value of the parenteral infusion composition is from about 4 to about 9. In some embodiments, the pH value of the parenteral infusion composition is from about 4 to about 8. In some embodiments, the pH value of the parenteral infusion composition is from about 5 to about 8.5. In some embodiments, the pH value of the parenteral infusion composition is from about 6 to about 7.5.

In some embodiments, 1 ml of the parenteral infusion composition comprises no more than 10 particles that are greater than 10 μm in size. In some embodiments, 1 ml of the parenteral infusion composition comprises no more than 20 particles that are greater than 10 m in size. In some embodiments, 1 ml of the parenteral infusion composition comprises no more than 30 particles that are greater than 10 μm in size. In some embodiments, 1 ml of the parenteral infusion composition comprises no more than 50 particles that are greater than 10 μm in size. In some embodiments, 1 ml of the parenteral infusion composition comprises no more than 100 particles that are greater than 10 μm in size. In some embodiments, 1 ml of the parenteral infusion composition comprises no more than 150 particles that are greater than 10 μm in size. In some embodiments, 1 ml of the parenteral infusion composition comprises no more than 200 particles that are greater than 10 μm in size. In some embodiments, 1 ml of the parenteral infusion composition comprises no more than 5 particles that are greater than 25 μm in size. In some embodiments, 1 ml of the parenteral infusion composition comprises no more than 10 particles that are greater than 25 μm in size. In some embodiments, 1 ml of the parenteral infusion composition comprises no more than 5 particles that are greater than 15 μm in size. In some embodiments, 1 ml of the parenteral infusion composition comprises no more than 20 particles that are greater than 25 μm in size.

In some embodiments, the concentration of cabazitaxel in the parenteral infusion composition is from about 0.01 mg/ml to about 0.5 mg/ml. In some embodiments, the concentration of cabazitaxel in the parenteral infusion composition is from about 0.02 mg/ml to about 0.25 mg/ml. In some embodiments, the concentration of cabazitaxel in the parenteral infusion composition is from about 0.03 mg/ml to about 0.2 mg/ml. In some embodiments, the concentration of cabazitaxel in the parenteral infusion composition is from about 0.04 mg/ml to about 0.15 mg/ml. In some embodiments, the concentration of cabazitaxel in the parenteral infusion composition is from about 0.04 mg/ml to about 0.1 mg/ml. In some embodiments, the concentration of cabazitaxel in the parenteral infusion composition is from about 0.05 mg/ml to about 0.2 mg/ml. In some embodiments, the concentration of cabazitaxel in the parenteral infusion composition is about 0.08 mg/ml or about 0.1 mg/ml. In some embodiments, weight ratio of human serum albumin to cabazitaxel is from about 10:1 to about 500:1. In some embodiments, weight ratio of human serum albumin to cabazitaxel is from about 50:1 to about 300:1. In some embodiments, weight ratio of human serum albumin to cabazitaxel is from about 10:1 to about 200:1. In some embodiments, weight ratio of human serum albumin to cabazitaxel is from about 20:1 to about 200:1. In some embodiments, weight ratio of human serum albumin to cabazitaxel is from about 50:1 to about 150:1. In some embodiments, weight ratio of human serum albumin to cabazitaxel is about 120:1, about 100:1, about 80:1, about 60:1, or about 50:1. In some embodiments, the concentration of human serum albumin in the parenteral infusion composition is from about 0.10% to about 20% (w/v). In some embodiments, the concentration of human serum albumin in the parenteral infusion composition is from about 0.25% to about 10% (w/v). In some embodiments, the concentration of human serum albumin in the parenteral infusion composition is from about 0.3% to about 8% (w/v). In some embodiments, the concentration of human serum albumin in the parenteral infusion composition is from about 0.3% to about 5% (w/v). In some embodiments, the concentration of human serum albumin in the parenteral infusion composition is from about 0.3% to about 2.5% (w/v). In some embodiments, the concentration of human serum albumin in the parenteral infusion composition is from about 0.3% to about 1.5% (w/v). In some embodiments, the concentration of human serum albumin in the parenteral infusion composition is from about 0.5% to about 1.2% (w/v). In some embodiments, the concentration of human serum albumin in the parenteral infusion composition is from about 0.5% to about 1% (w/v). In some embodiments, the concentration of human serum albumin in the parenteral infusion composition is from about 0.2% to about 1.5% (w/v).

In some embodiments, the first liquid composition comprises cabazitaxel, ethanol, and other organic solvents (e.g. propylene glycol, polyethylene glycol, hereinafter “PEG”, etc.). The PEG preferably has a molecular weight of about 300, i.e. PEG 300, or about 400, i.e. PEG 400. Other molecular weight PEG's known to those of ordinary skill can be included if desired in alternative embodiments. In some embodiments, the first liquid composition comprises cabazitaxel, ethanol, and propylene glycol. In some embodiments, the first liquid composition comprises cabazitaxel, ethanol, and PEG. In some embodiments, the first liquid composition comprises cabazitaxel, ethanol, and PEG 300. In some embodiments, the first liquid composition comprises cabazitaxel, ethanol, and PEG400. In some embodiments, the first liquid composition further comprises an acid. In some embodiments, the first liquid composition comprises cabazitaxel, ethanol, and an organic acid. The most preferred acid for use in accordance with the present invention is citric acid. In some embodiments, the first liquid composition comprises cabazitaxel, ethanol, and citric acid. In some embodiments, the first liquid composition comprises cabazitaxel, ethanol, propylene glycol, and citric acid. In some embodiments, the first liquid composition comprises cabazitaxel, ethanol, and citric acid. In some embodiments, the first liquid composition comprises cabazitaxel, ethanol, PEG (e.g. PEG 300, PEG 400), and citric acid. In some embodiments, the weight ratio of cabazitaxel and citric acid in the first liquid composition is from about 5000:1 to about 1:1. In some embodiments, the weight ratio of cabazitaxel and citric acid in the first liquid composition is from about 2000:1 to about 1:1. In some embodiments, the weight ratio of cabazitaxel and citric acid in the first liquid composition is from about 1000:1 to about 1:1. In some embodiments, the weight ratio of cabazitaxel and citric acid in the first liquid composition is from about 500:1 to about 1:1. In some embodiments, the weight ratio of cabazitaxel and citric acid in the first liquid composition is from about 5000:1 to about 5:1. In some embodiments, the weight ratio of cabazitaxel and citric acid in the first liquid composition is from about 2000:1 to about 5:1. In some embodiments, the weight ratio of cabazitaxel and citric acid in the first liquid composition is from about 1000:1 to about 5:1. In some embodiments, the weight ratio of cabazitaxel and citric acid in the first liquid composition is from about 500:1 to about 5:1. In some embodiments, the weight ratio of cabazitaxel and citric acid in the first liquid composition is from about 5000:1 to about 10:1. In some embodiments, the weight ratio of cabazitaxel and citric acid in the first liquid composition is from about 2000:1 to about 10:1. In some embodiments, the weight ratio of cabazitaxel and citric acid in the first liquid composition is from about 1000:1 to about 10:1. In some embodiments, the weight ratio of cabazitaxel and citric acid in the first liquid composition is from about 500:1 to about 10:1. In some embodiments, the weight ratio of cabazitaxel and citric acid in the first liquid composition is from about 2000:1 to about 100:1. In some embodiments, the weight ratio of cabazitaxel and citric acid in the first liquid composition is from about 1000:1 to about 100:1. In some embodiments, the weight ratio of cabazitaxel and citric acid in the first liquid composition is from about 1000:1 to about 20:1. In some embodiments, the weight ratio of cabazitaxel and citric acid in the first liquid composition is from about 500:1 to about 100:1. In some embodiments, the weight ratio of cabazitaxel and citric acid in the first liquid composition is from about 700:1 to about 200:1.

In some embodiments, concentration of cabazitaxel in the first liquid composition is from about 1 mg/ml to about 50 mg/ml. In some embodiments, concentration of cabazitaxel in the first liquid composition is from about 2 mg/ml to about 40 mg/ml. In some embodiments, concentration of cabazitaxel in the first liquid composition is from about 5 mg/ml to about 20 mg/ml. In some embodiments, concentration of cabazitaxel in the first liquid composition is about 10 mg/ml or about 5 mg/ml.

In some embodiments, the first liquid composition is prepared by dissolving cabazitaxel in a solvent comprising ethanol. In some embodiments, the first liquid composition is prepared by dissolving cabazitaxel and citric acid in a solvent comprising ethanol. In some embodiments, the first liquid composition is prepared by dissolving cabazitaxel and citric acid in ethanol. In some embodiments, the first liquid composition is prepared by dissolving cabazitaxel and citric acid in a solvent comprising ethanol and propylene glycol. In some embodiments, the first liquid composition is prepared by dissolving cabazitaxel and citric acid in a solvent comprising ethanol and PEG (e.g., PEG 300, PEG 400).

In some embodiments, the second aqueous composition is prepared by adding a human serum albumin solution into a parenterally acceptable vehicle. In some embodiments, the second aqueous composition is prepared by adding a human serum albumin solution into an infusion bag or bottle which contains a parenterally acceptable vehicle. In some embodiments, the second aqueous composition is prepared by adding a human serum albumin solution into a normal saline solution or dextrose solution. In some embodiments, the second aqueous composition is prepared by adding a human serum albumin solution into an infusion bag or bottle which contains a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a 20% solution of human serum albumin for infusion (w/v) into a normal saline solution or dextrose solution. In some embodiments, the second aqueous composition is prepared by adding a 25% solution of human serum albumin for infusion (w/v) into a normal saline solution or dextrose solution. In some embodiments, the second aqueous composition is prepared by adding a 5% solution of human serum albumin for infusion (w/v) into a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a clinically used solution of human serum albumin for infusion (e.g. 20%, 25%, 5%, w/v) into an infusion bag or bottle which contains a normal saline solution or dextrose solution. In some embodiments, the second aqueous composition is prepared by adding a 20% solution of human serum albumin for infusion (w/v) into an infusion bag or bottle which contains a normal saline solution or dextrose solution. In some embodiments, the second aqueous composition is prepared by adding a 25% solution of human serum albumin for infusion (w/v) into an infusion bag or bottle which contains a normal saline solution or dextrose solution. In some embodiments, the second aqueous composition is prepared by adding a 5% solution of human serum albumin for infusion (w/v) into an infusion bag or bottle which contains a normal saline solution or dextrose solution. In some embodiments, the second aqueous composition is an aqueous solution comprising human serum albumin. In some embodiments, the concentration of human serum albumin in the second aqueous composition is from about 0.10% to about 20% (w/v). In some embodiments, the concentration of human serum albumin in the second aqueous composition is from about 0.10% to about 10% (w/v). In some embodiments, the concentration of human serum albumin in the second aqueous composition is from about 0.2% to about 5% (w/v). In some embodiments, the concentration of human serum albumin in the second aqueous composition is from about 0.3% to about 2% (w/v). In some embodiments, the concentration of human serum albumin in the second aqueous composition is from about 0.3% to about 1.5% (w/v). In some embodiments, the concentration of human serum albumin in the second aqueous composition is from about 0.2% to about 1.5% (w/v). In some embodiments, the concentration of human serum albumin in the second aqueous composition is from about 0.4% to about 1% (w/v). In some embodiments, the second aqueous composition comprises from about 0.1 g to about 20 g of human serum albumin. In some embodiments, the second aqueous composition comprises from about 0.5 g to about 10 g of human serum albumin. In some embodiments, the second aqueous composition comprises from about 0.5 g to about 5 g of human serum albumin.

In some embodiments, the volume of the second aqueous composition is from about 100 ml to about 1 L. In some embodiments, the volume of the second aqueous composition is from about 100 ml to about 500 ml. In some embodiments, the volume of the second aqueous composition is from about 150 ml to about 500 ml. In some embodiments, the volume of the second aqueous composition is from about 150 ml to about 450 ml. In some embodiments, the volume of the second aqueous composition is from about 250 ml to about 500 ml. In some embodiments, the volume of the second aqueous composition is from about 250 ml to about 450 ml.

In some embodiments, the infusion composition of the present disclosure can be prepared by mixing the first liquid composition and the second aqueous composition. In some embodiments, the infusion composition can be prepared by injecting the first liquid composition into an infusion bag or bottle containing the second aqueous composition. The injection of the first liquid composition into the infusion bag or bottle containing the second aqueous composition is rapid. In some embodiments, the injection time is no more than 60 seconds. In some embodiments, the injection time is no more than 30 seconds. In some embodiments, the injection time is no more than 15 seconds. In some embodiments, the injection time is no more than 10 seconds. In some embodiments, the injection time is no more than 5 seconds. In some embodiments, the injection time is no more than 3 seconds. In some embodiments, the injection time is about 1-2 seconds. In some embodiments, the injection of the first liquid composition into the infusion bag or bottle containing the second aqueous composition is done at the temperature of the second aqueous composition at from about 15° C. to about 30° C. In some embodiments, the injection of the first liquid composition into the infusion bag or bottle containing the second aqueous composition is done at the temperature of the second aqueous composition at below about 30° C. In some embodiments, the injection of the first liquid composition into the infusion bag or bottle containing the second aqueous composition is done at the temperature of the second aqueous composition at from about 10° C. to about 25° C. In some embodiments, the injection of the first liquid composition into the infusion bag or bottle containing the second aqueous composition is done at the temperature of the second aqueous composition at below about 25° C. In some embodiments, the injection of the first liquid composition into the infusion bag or bottle containing the second aqueous composition is done at the temperature of the second aqueous composition at from about 5° C. to about 25° C.

In the process of injecting the first liquid composition into the infusion bag or bottle containing the second aqueous composition, mixing (e.g., manual mixing such as with a stirrer) or agitation is not need. After the injection of the first liquid composition into the infusion bag or bottle is completed, the first liquid composition and second aqueous composition are mixed well (e.g., the bag containing the composition is gently inverted by hand) to obtain a transparent infusion solution free of precipitates. In some embodiments, the infusion bag or bottle containing the second aqueous composition is held still in the injection process. In some embodiments, the first liquid composition is injected underneath the liquid surface of the second aqueous composition in the injection process. In some embodiments, after the injection is completed, the infusion bag or bottle is gently inverted repeatedly to mix well of the first liquid composition and second aqueous composition. In some embodiments, after the injection is completed, the infusion bag or bottle is gently inverted until a transparent solution free of precipitates is obtained. In some embodiments, after the injection is completed, the infusion bag or bottle is gently inverted for from about 5 seconds to about 10 minutes. In some embodiments, after the injection is completed, the infusion bag or bottle is gently inverted for from about 10 seconds to about 5 minutes. In some embodiments, after the injection is completed, the infusion bag or bottle is gently inverted for from about 0.5 minute to about 3 minutes. In some embodiments, after the injection is completed, the infusion bag or bottle is gently inverted for from about 15 seconds to about 2 minutes. In some embodiments, after the injection is completed, the infusion bag or bottle is gently inverted for from about 20 seconds to about 1.5 minutes. In some embodiments, after the injection is completed, the first liquid composition and second aqueous composition are mixed well until a transparent infusion solution free of precipitates is obtained.

After the first liquid composition and second aqueous composition are mixed well followed the injection of the first liquid composition into the infusion bag or bottle comprising the second aqueous composition, a transparent infusion solution free of precipitates comprising human serum albumin, cabazitaxel, and ethanol in a parenterally acceptable vehicle is obtained. In some embodiments, the transparent infusion solution further comprises citric acid. In some embodiments, a parenterally acceptable vehicle is a normal saline solution or dextrose solution. In some embodiments, the transparent infusion solution stays transparent free of visible particles or precipitates for at least about 1 hour. In some embodiments, the transparent infusion solution stays transparent free of visible particles or precipitates for at least about 2 hours. In some embodiments, the transparent infusion solution stays transparent free of visible particles or precipitates for at least about 3 hours. In some embodiments, the transparent infusion solution stays transparent free of visible particles or precipitates for at least about 4 hours. In some embodiments, the transparent infusion solution stays transparent free of visible particles or precipitates for at least about 6 hours. In some embodiments, the transparent infusion solution stays transparent free of visible particles or precipitates for at least about 8 hours. In some embodiments, the transparent infusion solution stays transparent free of visible particles or precipitates for at least about 24 hours. In some embodiments, the transparent infusion solution stays transparent free of visible particles or precipitates for at least about 1 hour, about 2 hours, about 3 hours, about 4 hours, about 8 hours, or about 12 hours, when the transparent solution is kept at from about 18° C. to about 25° C. In some embodiments, the transparent infusion solution stays transparent free of visible particles or precipitates for at least about 1 hour, about 2 hours, about 3 hours, about 4 hours, about 8 hours, or about 12 hours, when the transparent solution is kept at from about 20° C. to about 25° C. In some embodiments, the transparent solution stays transparent free of visible particles or precipitates for at least about 2 hours, about 4 hours, about 6 hours, about 8 hours, about 12 hours, or about 24 hours, when the transparent solution is kept at about 2-8° C.

Also, provided herein is a kit for the preparation of a parenteral infusion solution comprising: a first container containing a composition comprising cabazitaxel; and a second container containing a composition comprising human serum albumin. In some embodiments, the kit comprises a first container containing a liquid composition comprising cabazitaxel, and a second container containing a liquid composition comprising human serum albumin. In some embodiments, the kit comprises a first container containing a liquid composition comprising cabazitaxel and ethanol, and a second container containing an aqueous composition comprising human serum albumin. In some embodiments, the kit comprises a first container containing a liquid composition comprising cabazitaxel, citric acid, and ethanol, and a second container containing an aqueous composition comprising human serum albumin. In some embodiments, the kit comprises a first container containing a liquid composition comprising cabazitaxel, citric acid, and dehydrated ethanol, and a second container containing an aqueous composition comprising human serum albumin. In some embodiments, the kit comprises a first container containing a liquid composition comprising cabazitaxel, citric acid, and ethanol, and a second container containing a solution of human serum albumin for infusion (e.g. about 20%, about 25%, about 5%, about 2%, about 1.5%, or about 1% (w/v)).

In some embodiments, the first container does not comprise a lipid (e.g. soybean oil). In some embodiments, the second container does not comprise a lipid (e.g. soybean oil). In some embodiments, the lipid is soybean oil. In some embodiments, the first container does not comprise a surfactant. In some embodiments, the second container does not comprise a surfactant. In some embodiments, neither the first container, nor the second container comprises Polysorbate 80.

In some embodiments, the first container contains a liquid composition comprising cabazitaxel and ethanol. In some embodiments, the first container contains a liquid composition comprising cabazitaxel, an acid, and ethanol. In some embodiments, the first container contains a liquid composition comprising cabazitaxel, an organic acid, and ethanol. In some embodiments, the first container contains a liquid composition comprising cabazitaxel, ethanol, and other organic solvents (e.g., alcohols such as propylene glycol, polyethylene glycol 300, etc.). In some embodiments, the first container contains a liquid composition comprising cabazitaxel, ethanol, and propylene glycol. In some embodiments, the first container contains a liquid composition comprising cabazitaxel, ethanol, and polyethylene glycol 300. In some embodiments, the first container contains a liquid composition comprising cabazitaxel, ethanol, and polyethylene glycol 400. In some embodiments, the first container contains a liquid composition comprising cabazitaxel and citric acid. In some embodiments, the first container contains a liquid composition comprising cabazitaxel, citric acid, and ethanol. In some embodiments, the first container contains a liquid composition comprising cabazitaxel, citric acid, ethanol, and propylene glycol. In some embodiments, the weight ratio of cabazitaxel and citric acid in the first container is from about 5000:1 to about 1:1. In some embodiments, the weight ratio of cabazitaxel and citric acid in the first container is from about 2000:1 to about 1:1. In some embodiments, the weight ratio of cabazitaxel and citric acid in the first container is from about 1000:1 to about 1:1. In some embodiments, the weight ratio of cabazitaxel and citric acid in the first container is from about 500:1 to about 1:1. In some embodiments, the weight ratio of cabazitaxel and citric acid in the first container is from about 5000:1 to about 5:1. In some embodiments, the weight ratio of cabazitaxel and citric acid in the first container is from about 2000:1 to about 5:1. In some embodiments, the weight ratio of cabazitaxel and citric acid in the first container is from about 1000:1 to about 5:1. In some embodiments, the weight ratio of cabazitaxel and citric acid in the first container is from about 500:1 to about 5:1. In some embodiments, the weight ratio of cabazitaxel and citric acid in the first container is from about 5000:1 to about 10:1. In some embodiments, the weight ratio of cabazitaxel and citric acid in the first container is from about 2000:1 to about 10:1. In some embodiments, the weight ratio of cabazitaxel and citric acid in the first container is from about 1000:1 to about 10:1. In some embodiments, the weight ratio of cabazitaxel and citric acid in the first container is from about 500:1 to about 10:1. In some embodiments, the weight ratio of cabazitaxel and citric acid in the first container is from about 2000:1 to about 100:1. In some embodiments, the weight ratio of cabazitaxel and citric acid in the first container is from about 1000:1 to about 100:1. In some embodiments, the weight ratio of cabazitaxel and citric acid in the first container is from about 1000:1 to about 20:1. In some embodiments, the weight ratio of cabazitaxel and citric acid in the first container is from about 500:1 to about 50:1. In some embodiments, the weight ratio of cabazitaxel and citric acid in the first container is from about 700:1 to about 200:1.

In some embodiments, the amount of cabazitaxel contained in the first container is from about 5 mg to about 100 mg of cabazitaxel. In some embodiments, the amount of cabazitaxel contained in the first container is from about 5 mg to about 80 mg of cabazitaxel. In some embodiments, the amount of cabazitaxel contained in the first container is from about 10 mg to about 60 mg of cabazitaxel. In some embodiments, the amount of cabazitaxel contained in the first container is from about 10 mg to about 50 mg of cabazitaxel. In some embodiments, the amount of cabazitaxel contained in the first container is about 10 mg, about 20 mg, about 30 mg, about 40 mg, about 50 mg, or about 60 mg of cabazitaxel. In some embodiments, the concentration of cabazitaxel in the first container is from about 1 mg/ml to about 50 mg/ml. In some embodiments, the concentration of cabazitaxel in the first container is from about 5 mg/ml to about 40 mg/ml. In some embodiments, the concentration of cabazitaxel in the first container is from about 5 mg/ml to about 20 mg/ml. In some embodiments, the concentration of cabazitaxel in the first container is about 5 mg/ml or about 10 mg/ml. In some embodiments, the amount of ethanol contained in the first container is from about 0.5 ml to about 50 ml of ethanol. In some embodiments, the amount of ethanol contained in the first container is from about 1 ml to about 20 ml of ethanol. In some embodiments, the amount of ethanol contained in the first container is from about 1 ml to about 10 ml of ethanol. In some embodiments, the amount of ethanol contained in the first container is from about 1 ml to about 6 ml of ethanol. In some embodiments, the amount of ethanol contained in the first container is from about 1 ml to about 5 ml of ethanol.

In some embodiments, the liquid composition in the first container is prepared by dissolving cabazitaxel in ethanol. In some embodiments, the liquid composition in the first container is prepared by dissolving cabazitaxel and citric acid in ethanol. In some embodiments, the liquid composition in the first container is prepared by dissolving cabazitaxel in a solvent comprising ethanol. In some embodiments, the liquid composition in the first container is prepared by dissolving cabazitaxel and citric acid in a solvent comprising ethanol.

In some embodiments, the first container contains a solid composition comprising cabazitaxel. In some embodiments, the first container contains a solid composition comprising cabazitaxel and an organic acid. In some embodiments, the first container contains a solid composition comprising cabazitaxel and citric acid.

In some embodiments, the second container contains an aqueous composition comprising human serum albumin. In some embodiments, the second container contains a human serum albumin solution. In some embodiments, the second container contains a human serum albumin solution for infusion. In some embodiments, the second container contains a human serum albumin solution with the concentration of human serum albumin in the solution from about 1% to about 25% (w/v). In some embodiments, the second container contains a human serum albumin solution with the concentration of human serum albumin in the solution from about 5% to about 25% (w/v). In some embodiments, the second container contains a solution of human serum albumin for infusion. In some embodiments, the second container contains a 20% solution of human serum albumin for infusion (w/v). In some embodiments, the second container contains a 20% solution of human serum albumin for infusion USP (w/v). In some embodiments, the second container contains a 25% solution of human serum albumin for infusion (w/v). In some embodiments, the second container contains a 25% solution of human serum albumin for infusion USP (w/v). In some embodiments, the second container contains a 5% solution of human serum albumin for infusion (w/v). In some embodiments, the second container contains a 5% solution of human serum albumin for infusion USP (w/v). In some embodiments, the second container contains a liquid composition comprising from about 1 g to about 50 g of human serum albumin. In some embodiments, the second container contains a liquid composition comprising from about 1 g to about 20 g of human serum albumin. In some embodiments, the second container contains a liquid composition comprising from about 1 g to about 10 g of human serum albumin. In some embodiments, the second container contains a liquid composition comprising from about 2 g to about 8 g of human serum albumin.

In some embodiments, the kit further comprises instructions to add the composition comprising cabazitaxel to the composition comprising human serum albumin.

The preparation of the parenteral infusion solution from the composition comprising cabazitaxel in the first container and the composition comprising human serum albumin in the second container follows the methods as disclosed in the current invention. In some embodiments, the liquid composition comprising human serum albumin form the second container is added into an infusion bag or bottle, followed by injecting the liquid composition comprising cabazitaxel from the first container into the infusion bag or bottle. In some embodiments, the liquid composition comprising cabazitaxel from the first container is injected into the second container comprising the liquid composition comprising human serum albumin to prepare the parenteral infusion solution.

Also, provided herein is a liquid composition comprising cabazitaxel and ethanol, wherein the composition does not contain a surfactant (e.g. Polysorbate 80), and wherein the said liquid composition is mixed with a second aqueous composition comprising human serum albumin to form an infusion solution comprising cabazitaxel prior to being infused or administered to patients. In some embodiments, the liquid composition is an injectable pharmaceutical composition. In some embodiments, the liquid composition further comprises citric acid. In some embodiments, the two compositions are mixed to form an infusion solution in less than or equal to about 24 hours prior to being infused or administered to patients. In some embodiments, the two compositions are mixed to form an infusion solution in less than or equal to about 8 hours prior to being infused or administered to patients. In some embodiments, the two compositions are mixed to form an infusion solution in less than or equal to about 6 hours prior to being infused or administered to patients. In some embodiments, the two compositions are mixed to form an infusion solution in less than or equal to about 4 hours prior to being infused or administered to patients. In some embodiments, the two compositions are mixed to form an infusion solution in less than or equal to about 2 hours prior to being infused or administered to patients. In some embodiments, the two compositions are mixed to form an infusion solution in less than or equal to about 1 hour prior to being infused or administered to patients. In some embodiments, the two compositions are mixed to form an infusion solution right before being infused or administered to patients. In some embodiments, the mixing is done in an infusion bag or infusion bottle. In some embodiments, the second aqueous composition comprises a parenterally acceptable vehicle. In some embodiments, the second aqueous composition is contained in an infusion bag or infusion bottle. In some embodiments, the liquid composition is injected into an infusion bag or infusion bottle which contains the second aqueous composition comprising human serum albumin and a parenterally acceptable vehicle. In some embodiments, mixing (e.g., manual mixing such as with a stirrer) or agitation is not need in the process of injecting the first liquid composition into an infusion bag or bottle which contains the second aqueous composition.

In some embodiments, the liquid composition does not comprise a lipid (e.g. soybean oil). In some embodiments, the lipid is soybean oil.

In some embodiments, the liquid composition comprises cabazitaxel, ethanol, and other organic solvents (e.g. propylene glycol, polyethylene glycol, hereinafter “PEG”, etc.). The PEG preferably has a molecular weight of about 300, i.e. PEG 300, or about 400, i.e. PEG 400. Other molecular weight PEG's known to those of ordinary skill can be included if desired in alternative embodiments. In some embodiments, the liquid composition comprises cabazitaxel, ethanol, and propylene glycol. In some embodiments, the liquid composition comprises cabazitaxel, ethanol, and PEG. In some embodiments, the liquid composition comprises cabazitaxel, ethanol, and PEG 300. In some embodiments, the liquid composition comprises cabazitaxel, ethanol, and PEG400. In some embodiments, the liquid composition further comprises an acid. In some embodiments, the liquid composition comprises cabazitaxel, ethanol, and an organic acid. The most preferred acid for use in accordance with the present invention is citric acid. In some embodiments, the liquid composition comprises cabazitaxel, ethanol, and citric acid. In some embodiments, the liquid composition comprises cabazitaxel, ethanol, propylene glycol, and citric acid. In some embodiments, the liquid composition comprises cabazitaxel, ethanol, PEG (e.g. PEG 300, PEG 400), and citric acid. In some embodiments, the weight ratio of cabazitaxel and citric acid in the liquid composition is from about 5000:1 to about 1:1. In some embodiments, the weight ratio of cabazitaxel and citric acid in the liquid composition is from about 2000:1 to about 1:1. In some embodiments, the weight ratio of cabazitaxel and citric acid in the liquid composition is from about 1000:1 to about 1:1. In some embodiments, the weight ratio of cabazitaxel and citric acid in the liquid composition is from about 500:1 to about 1:1. In some embodiments, the weight ratio of cabazitaxel and citric acid in the liquid composition is from about 5000:1 to about 5:1. In some embodiments, the weight ratio of cabazitaxel and citric acid in the liquid composition is from about 2000:1 to about 5:1. In some embodiments, the weight ratio of cabazitaxel and citric acid in the liquid composition is from about 1000:1 to about 5:1. In some embodiments, the weight ratio of cabazitaxel and citric acid in the liquid composition is from about 500:1 to about 5:1. In some embodiments, the weight ratio of cabazitaxel and citric acid in the liquid composition is from about 5000:1 to about 10:1. In some embodiments, the weight ratio of cabazitaxel and citric acid in the liquid composition is from about 2000:1 to about 10:1. In some embodiments, the weight ratio of cabazitaxel and citric acid in the liquid composition is from about 1000:1 to about 10:1. In some embodiments, the weight ratio of cabazitaxel and citric acid in the liquid composition is from about 500:1 to about 10:1. In some embodiments, the weight ratio of cabazitaxel and citric acid in the liquid composition is from about 2000:1 to about 100:1. In some embodiments, the weight ratio of cabazitaxel and citric acid in the liquid composition is from about 1000:1 to about 100:1. In some embodiments, the weight ratio of cabazitaxel and citric acid in the liquid composition is from about 1000:1 to about 50:1. In some embodiments, the weight ratio of cabazitaxel and citric acid in the liquid composition is from about 500:1 to about 50:1. In some embodiments, the weight ratio of cabazitaxel and citric acid in the liquid composition is from about 700:1 to about 200:1.

In some embodiments, the liquid composition comprises cabazitaxel and a solvent comprising ethanol, wherein the cabazitaxel is dissolved in the solvent. In some embodiments, the liquid composition comprises cabazitaxel, an acid, and a solvent comprising ethanol, wherein the cabazitaxel and the acid are dissolved in the solvent. In some embodiments, the liquid composition comprises cabazitaxel, an organic acid, and a solvent comprising ethanol, wherein the cabazitaxel and the organic acid are dissolved in the solvent. In some embodiments, the liquid composition comprises cabazitaxel, citric acid, and a solvent comprising ethanol, wherein the cabazitaxel and citric acid are dissolved in the solvent. In some embodiments, the liquid composition comprises cabazitaxel and ethanol, wherein the cabazitaxel is dissolved in ethanol. In some embodiments, the liquid composition comprises cabazitaxel, citric acid, and ethanol, wherein the cabazitaxel and citric acid are dissolved in ethanol.

In some embodiments, the amount of cabazitaxel in the liquid composition is from about 5 mg to about 100 mg of cabazitaxel. In some embodiments, the amount of cabazitaxel in the liquid composition is from about 5 mg to about 80 mg of cabazitaxel. In some embodiments, the amount of cabazitaxel in the liquid composition is from about 10 mg to about 60 mg of cabazitaxel. In some embodiments, the amount of cabazitaxel in the liquid composition is from about 10 mg to about 50 mg of cabazitaxel. In some embodiments, the amount of cabazitaxel in the liquid composition is about 10 mg, about 20 mg, about 30 mg, about 40 mg, about 50 mg, or about 60 mg of cabazitaxel. In some embodiments, the concentration of cabazitaxel in the liquid composition is from about 1 mg/ml to about 50 mg/ml. In some embodiments, the concentration of cabazitaxel in the liquid composition is from about 5 mg/ml to about 40 mg/ml. In some embodiments, the concentration of cabazitaxel in the liquid composition is from about 5 mg/ml to about 20 mg/ml. In some embodiments, the concentration of cabazitaxel in the liquid composition is about 5 mg/ml or about 10 mg/ml. In some embodiments, the amount of ethanol in the liquid composition is from about 0.5 ml to about 50 ml of ethanol. In some embodiments, the amount of ethanol in the liquid composition is from about 1 ml to about 20 ml of ethanol. In some embodiments, the amount of ethanol in the liquid composition is from about 1 ml to about 10 ml of ethanol. In some embodiments, the amount of ethanol in the liquid composition is from about 1 ml to about 6 ml of ethanol. In some embodiments, the amount of ethanol in the liquid composition is from about 1 ml to about 5 ml of ethanol.

In some embodiments, the liquid composition is prepared by dissolving cabazitaxel, in a solvent comprising ethanol. In some embodiments, the liquid composition is prepared by dissolving cabazitaxel, in ethanol. In some embodiments, the liquid composition is prepared by dissolving cabazitaxel and citric acid in a solvent comprising ethanol. In some embodiments, the liquid composition is prepared by dissolving cabazitaxel and citric acid in ethanol. In some embodiments, the liquid composition is prepared by dissolving cabazitaxel and citric acid in a solvent comprising ethanol and propylene glycol. In some embodiments, the liquid composition is prepared by dissolving cabazitaxel and citric acid in a solvent comprising ethanol and PEG (e.g. PEG 300, PEG 400).

In some embodiments, pH of the solution obtained by mixing the liquid composition and water in 1:1 (v/v) ratio is from about 3 to about 7. In some embodiments, pH of the solution obtained by mixing the liquid composition and water in 1:1 (v/v) ratio is from about 3 to about 6. In some embodiments, pH of the solution obtained by mixing the liquid composition and water in 1:1 (v/v) ratio is from about 4 to about 6. In some embodiments, pH of the solution obtained by mixing the liquid composition and water in 1:1 (v/v) ratio is about 5.

In some embodiments, the second aqueous composition is prepared by adding a human serum albumin solution into a parenterally acceptable vehicle. In some embodiments, the second aqueous composition is prepared by adding a human serum albumin solution into an infusion bag or bottle which contains a parenterally acceptable vehicle. In some embodiments, the second aqueous composition is prepared by adding a human serum albumin solution into a normal saline solution or dextrose solution. In some embodiments, the second aqueous composition is prepared by adding a human serum albumin solution into an infusion bag or bottle which contains a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a solution of human serum albumin for infusion into a normal saline solution or dextrose solution. In some embodiments, the second aqueous composition is prepared by adding a 20% solution of human serum albumin for infusion (w/v) into a normal saline solution or dextrose solution. In some embodiments, the second aqueous composition is prepared by adding a 25% solution of human serum albumin for infusion (w/v) into a normal saline solution or dextrose solution. In some embodiments, the second aqueous composition is prepared by adding a 5% solution of human serum albumin for infusion (w/v) into a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a solution of human serum albumin for infusion into an infusion bag or bottle which contains a normal saline solution or dextrose solution. In some embodiments, the second aqueous composition is prepared by adding a 20% solution of human serum albumin for infusion (w/v) into an infusion bag or bottle which contains a normal saline solution or dextrose solution. In some embodiments, the second aqueous composition is prepared by adding a 25% solution of human serum albumin for infusion (w/v) into an infusion bag or bottle which contains a normal saline solution or dextrose solution. In some embodiments, the second aqueous composition is prepared by adding a 5% solution of human serum albumin for infusion (w/v) into an infusion bag or bottle which contains a normal saline solution or dextrose solution. In some embodiments, the second aqueous composition is an aqueous solution comprising human serum albumin. In some embodiments, the concentration of human serum albumin in the second aqueous composition is from about 0.1% to about 20% (w/v). In some embodiments, the concentration of human serum albumin in the second aqueous composition is from about 0.1% to about 10% (w/v). In some embodiments, the concentration of human serum albumin in the second aqueous composition is from 0.2% to 5% (w/v). In some embodiments, the concentration of human serum albumin in the second aqueous composition is from 0.5% to 2% (w/v). In some embodiments, the concentration of human serum albumin in the second aqueous composition is from 0.5% to 1.5% (w/v). In some embodiments, the second aqueous composition comprises from about 0.1 g to about 20 g of human serum albumin. In some embodiments, the second aqueous composition comprises from about 0.5 g to about 10 g of human serum albumin. In some embodiments, the second aqueous composition comprises from about 0.5 g to about 5 g of human serum albumin.

The injection of the liquid composition into the infusion bag or bottle containing the second aqueous composition is rapid. In some embodiments, the injection time is no more than 60 seconds. In some embodiments, the injection time is no more than 45 seconds. In some embodiments, the injection time is no more than 30 seconds. In some embodiments, the injection time is no more than 15 seconds. In some embodiments, the injection time is no more than 10 seconds. In some embodiments, the injection time is no more than 5 seconds.

In the process of injecting the liquid composition into the infusion bag or bottle containing the second aqueous composition, mixing or agitation is not need. After the injection of the liquid composition into the infusion bag or bottle is completed, the liquid composition and second aqueous composition are mixed well (e.g., the bag containing the composition is gently inverted by hand) to obtain a transparent infusion solution free of precipitates. In some embodiments, the infusion bag or bottle containing the second aqueous composition is held still in the injection process. In some embodiments, the liquid composition is injected underneath the liquid surface of the second aqueous composition in the injection process. In some embodiments, after the injection is completed, the infusion bag or bottle is gently inverted repeatedly to mix well of the first liquid composition and second aqueous composition. In some embodiments, after the injection is completed, the infusion bag or bottle is gently inverted until a transparent solution free of precipitates is obtained. In some embodiments, after the injection is completed, the infusion bag or bottle is gently inverted for from about 5 seconds to about 10 minutes. In some embodiments, after the injection is completed, the infusion bag or bottle is gently inverted for from about 10 seconds to about 5 minutes. In some embodiments, after the injection is completed, the infusion bag or bottle is gently inverted for from about 0.5 minute to about 3 minutes. In some embodiments, after the injection is completed, the first liquid composition and second aqueous composition are mixed well until a transparent infusion solution free of precipitates is obtained.

Also provided in the present invention is a composition comprising cabazitaxel and albumin, wherein the composition is an aqueous solution, wherein at least 10% cabazitaxel in the composition is Free (unbound in solution) cabazitaxel, and wherein the concentration of albumin in the composition is from about 0.10% to about 25% (w/v). In some embodiments, albumin in the composition is serum albumin. In some embodiments, albumin in the composition is human serum albumin. In some embodiments, the composition is a clear aqueous solution. In some embodiments, the composition comprises a pharmaceutically acceptable vehicle (e.g. a normal saline solution or dextrose solution). In some embodiments, the composition comprises ethanol. In some embodiments, the composition comprises a pharmaceutically acceptable vehicle and ethanol.

In some embodiments, at least 20% cabazitaxel in the composition is Free (unbound in solution) cabazitaxel. In some embodiments, at least 30% cabazitaxel in the composition is Free (unbound in solution) cabazitaxel. In some embodiments, at least 40% cabazitaxel in the composition is Free (unbound in solution) cabazitaxel. In some embodiments, at least 50% cabazitaxel in the composition is Free (unbound in solution) cabazitaxel. In some embodiments, at least 60% cabazitaxel in the composition is Free (unbound in solution) cabazitaxel. In some embodiments, at least 70% cabazitaxel in the composition is Free (unbound in solution) cabazitaxel. In some embodiments, at least 80% cabazitaxel in the composition is Free (unbound in solution) cabazitaxel. In some embodiments, from about 10% to about 80% of cabazitaxel in the composition is Free (unbound in solution) cabazitaxel. In some embodiments, from about 10% to about 70% of cabazitaxel in the composition is Free (unbound in solution) cabazitaxel. In some embodiments, from about 10% to about 60% of cabazitaxel in the composition is Free (unbound in solution) cabazitaxel. In some embodiments, from about 10% to about 50% of cabazitaxel in the composition is Free (unbound in solution) cabazitaxel. In some embodiments, from about 20% to about 80% of cabazitaxel in the composition is Free (unbound in solution) cabazitaxel. In some embodiments, from about 20% to about 70% of cabazitaxel in the composition is Free (unbound in solution) cabazitaxel. In some embodiments, from about 20% to about 60% of cabazitaxel in the composition is Free (unbound in solution) cabazitaxel. In some embodiments, from about 20% to about 50% of cabazitaxel in the composition is Free (unbound in solution) cabazitaxel. In some embodiments, from about 30% to about 80% of cabazitaxel in the composition is Free (unbound in solution) cabazitaxel. In some embodiments, from about 30% to about 70% of cabazitaxel in the composition is Free (unbound in solution) cabazitaxel. In some embodiments, from about 30% to about 60% of cabazitaxel in the composition is Free (unbound in solution) cabazitaxel. In some embodiments, from about 10% to about 55% of cabazitaxel in the composition is Free (unbound in solution) cabazitaxel. In some embodiments, from about 40% to about 80% of cabazitaxel in the composition is Free (unbound in solution) cabazitaxel. In some embodiments, from about 40% to about 70% of cabazitaxel in the composition is Free (unbound in solution) cabazitaxel. In some embodiments, from about 40% to about 65% of cabazitaxel in the composition is Free (unbound in solution) cabazitaxel. In some embodiments, from about 45% to about 70% of cabazitaxel in the composition is Free (unbound in solution) cabazitaxel. In some embodiments, from about 45% to about 65% of cabazitaxel in the composition is Free (unbound in solution) cabazitaxel. In some embodiments, from about 50% to about 80% of cabazitaxel in the composition is Free (unbound in solution) cabazitaxel. In some embodiments, from about 50% to about 70% of cabazitaxel in the composition is Free (unbound in solution) cabazitaxel.

Fraction of free (unbound in solution) cabazitaxel in the composition is measured by ultrafiltration through a 30-kDa membrane. The experiments of free (unbound in solution) cabazitaxel measurement are described in the example section.

In some embodiments, the concentration of albumin in the composition is from about 0.2% to about 20% (w/v). In some embodiments, the concentration of albumin in the composition is from about 0.5% to about 15% (w/v). In some embodiments, the concentration of albumin in the composition is from about 0.5% to about 10% (w/v). In some embodiments, the concentration of albumin in the composition is from about 1% to about 10% (w/v). In some embodiments, the concentration of albumin in the composition is from about 0.5% to about 5% (w/v). In some embodiments, the concentration of albumin in the composition is from about 5% to about 10% (w/v). In some embodiments, the concentration of albumin in the composition is from about 1% to about 5% (w/v). In some embodiments, the concentration of albumin in the composition is from about 0.3% to about 3% (w/v). In some embodiments, the concentration of albumin in the composition is from about 2% to about 6% (w/v). In some embodiments, the concentration of albumin in the composition is from about 0.5% to about 4% (w/v).

In some embodiments, the concentration of human serum albumin in the composition is from about 0.2% to about 20% (w/v). In some embodiments, the concentration of human serum albumin in the composition is from about 0.5% to about 15% (w/v). In some embodiments, the concentration of human serum albumin in the composition is from about 0.5% to about 10% (w/v). In some embodiments, the concentration of human serum albumin in the composition is from about 1% to about 10% (w/v). In some embodiments, the concentration of human serum albumin in the composition is from about 0.5% to about 5% (w/v). In some embodiments, the concentration of human serum albumin in the composition is from about 5% to about 10% (w/v). In some embodiments, the concentration of human serum albumin in the composition is from about 1% to about 5% (w/v). In some embodiments, the concentration of human serum albumin in the composition is from about 0.3% to about 3% (w/v). In some embodiments, the concentration of human serum albumin in the composition is from about 2% to about 6% (w/v). In some embodiments, the concentration of human serum albumin in the composition is from about 0.5% to about 4% (w/v).

In some embodiments, the composition comprises ethanol. In some embodiments, the amount of ethanol in the composition is no more than 15% (v/v). In some embodiments, the amount of ethanol in the composition is no more than 10% (v/v). In some embodiments, the amount of ethanol in the composition is no more than 5% (v/v). In some embodiments, the amount of ethanol in the composition is no more than 3% (v/v). In some embodiments, the amount of ethanol in the composition is from about 0.1% to about 15% (v/v). In some embodiments, the amount of ethanol in the composition is from about 0.2% to about 10% (v/v). In some embodiments, the amount of ethanol in the composition is from about 0.10% to about 10% (v/v). In some embodiments, the amount of ethanol in the composition is from about 0.5% to about 3% (v/v). In some embodiments, the amount of ethanol in the composition is from about 0.1% to about 3% (v/v). In some embodiments, the amount of ethanol in the composition is from about 1% to about 5% (v/v). In some embodiments, the amount of ethanol in the composition is from about 0.1% to about 5% (v/v). In some embodiments, the amount of ethanol in the composition is from about 0.5% to about 5% (v/v). In some embodiments, the amount of ethanol in the composition is from about 0.5% to about 10% (v/v). In some embodiments, the amount of ethanol in the composition is from about 0.5% to about 3% (v/v). In some embodiments, the amount of ethanol in the composition is from about 2% to about 8% (v/v).

In some embodiments, the concentration of cabazitaxel in the composition is from about 0.01 mg/ml to about 1 mg/ml. In some embodiments, the concentration of cabazitaxel in the composition is from about 0.01 mg/ml to about 0.5 mg/ml. In some embodiments, the concentration of cabazitaxel in the composition is from about 0.01 mg/ml to about 0.3 mg/ml. In some embodiments, the concentration of cabazitaxel in the composition is from about 0.01 mg/ml to about 0.2 mg/ml. In some embodiments, the concentration of cabazitaxel in the composition is from about 0.03 mg/ml to about 1 mg/ml. In some embodiments, the concentration of cabazitaxel in the composition is from about 0.03 mg/ml to about 0.5 mg/ml. In some embodiments, the concentration of cabazitaxel in the composition is from about 0.03 mg/ml to about 0.3 mg/ml. In some embodiments, the concentration of cabazitaxel in the composition is from about 0.03 mg/ml to about 0.2 mg/ml. In some embodiments, the concentration of cabazitaxel in the composition is from about 0.05 mg/ml to about 1 mg/ml. In some embodiments, the concentration of cabazitaxel in the composition is from about 0.05 mg/ml to about 0.5 mg/ml. In some embodiments, the concentration of cabazitaxel in the composition is from about 0.05 mg/ml to about 0.3 mg/ml. In some embodiments, the concentration of cabazitaxel in the composition is from about 0.05 mg/ml to about 0.2 mg/ml. In some embodiments, the concentration of cabazitaxel in the composition is from about 0.07 mg/ml to about 1 mg/ml. In some embodiments, the concentration of cabazitaxel in the composition is from about 0.07 mg/ml to about 0.5 mg/ml. In some embodiments, the concentration of cabazitaxel in the composition is from about 0.07 mg/ml to about 0.3 mg/ml. In some embodiments, the concentration of cabazitaxel in the composition is from about 0.07 mg/ml to about 0.2 mg/ml. In some embodiments, the concentration of cabazitaxel in the composition is from about 0.1 mg/ml to about 1 mg/ml. In some embodiments, the concentration of cabazitaxel in the composition is from about 0.1 mg/ml to about 0.5 mg/ml. In some embodiments, the concentration of cabazitaxel in the composition is from about 0.1 mg/ml to about 0.3 mg/ml. In some embodiments, the concentration of cabazitaxel in the composition is from about 0.1 mg/ml to about 0.2 mg/ml. In some embodiments, the concentration of cabazitaxel in the composition is about 0.05 mg/ml, about 0.06 mg/ml, about 0.07 mg/ml, about 0.08 mg/ml, about 0.09 mg/ml, about 0.10 mg/ml, about 0.11 mg/ml, about 0.12 mg/ml, about 0.13 mg/ml, about 0.14 mg/ml, about 0.15 mg/ml, about 0.16 mg/ml, about 0.17 mg/ml, about 0.18 mg/ml, about 0.19 mg/ml, about 0.20 mg/ml, about 0.25 mg/ml, or about 0.30 mg/ml.

In some embodiments, the concentration of Free (unbound in solution) cabazitaxel in the composition is from about 0.001 mg/ml to about 0.2 mg/ml. In some embodiments, the concentration of Free (unbound in solution) cabazitaxel in the composition is from about 0.005 mg/ml to about 0.2 mg/ml. In some embodiments, the concentration of Free (unbound in solution) cabazitaxel in the composition is from about 0.01 mg/ml to about 0.2 mg/ml. In some embodiments, the concentration of Free (unbound in solution) cabazitaxel in the composition is from about 0.02 mg/ml to about 0.2 mg/ml. In some embodiments, the concentration of Free (unbound in solution) cabazitaxel in the composition is from about 0.002 mg/ml to about 0.15 mg/ml. In some embodiments, the concentration of Free (unbound in solution) cabazitaxel in the composition is from about 0.005 mg/ml to about 0.15 mg/ml. In some embodiments, the concentration of Free (unbound in solution) cabazitaxel in the composition is from about 0.01 mg/ml to about 0.15 mg/ml. In some embodiments, the concentration of Free (unbound in solution) cabazitaxel in the composition is from about 0.02 mg/ml to about 0.1 5 mg/ml. In some embodiments, the concentration of Free (unbound in solution) cabazitaxel in the composition is from about 0.005 mg/ml to about 0.12 mg/ml. In some embodiments, the concentration of Free (unbound in solution) cabazitaxel in the composition is from about 0.01 mg/ml to about 0.12 mg/ml. In some embodiments, the concentration of Free (unbound in solution) cabazitaxel in the composition is from about 0.02 mg/ml to about 0.12 mg/ml. In some embodiments, the concentration of Free (unbound in solution) cabazitaxel in the composition is from about 0.002 mg/ml to about 0.10 mg/ml. In some embodiments, the concentration of Free (unbound in solution) cabazitaxel in the composition is from about 0.005 mg/ml to about 0.10 mg/ml. In some embodiments, the concentration of Free (unbound in solution) cabazitaxel in the composition is from about 0.01 mg/ml to about 0.10 mg/ml. In some embodiments, the concentration of Free (unbound in solution) cabazitaxel in the composition is from about 0.02 mg/ml to about 0.10 mg/ml. In some embodiments, the concentration of Free (unbound in solution) cabazitaxel in the composition is from about 0.02 mg/ml to about 0.08 mg/ml. In some embodiments, the concentration of Free (unbound in solution) cabazitaxel in the composition is from about 0.03 mg/ml to about 0.08 mg/ml. In some embodiments, the concentration of Free (unbound in solution) cabazitaxel in the composition is from about 0.03 mg/ml to about 0.06 mg/ml. In some embodiments, the concentration of Free (unbound in solution) cabazitaxel in the composition is about 0.020 mg/ml, about 0.025 mg/ml, about 0.030 mg/ml, about 0.035 mg/ml, about 0.040 mg/ml, about 0.045 mg/ml, about 0.050 mg/ml, about 0.055 mg/ml, about 0.060 mg/ml, about 0.065 mg/ml, about 0.070 mg/ml, about 0.075 mg/ml, about 0.080 mg/ml, about 0.085 mg/ml, about 0.090 mg/ml, about 0.095 mg/ml, or about 0.10 mg/ml.

In some embodiments, the composition comprises pH adjusting agent(s). Suitable pH adjusting agents include, but are not limited to, diethanolamine, triethanolamine, sodium hydroxide, hydrochloric acid, citric acid and monobasic sodium phosphate and the like or their mixtures thereof. In some embodiments, pH adjusting agent is citric acid. In some embodiments, the pH value of the composition is from about 4 to about 9.5. In some embodiments, the pH value of the composition is from about 5 to about 9. In some embodiments, the pH value of the composition is from about 5 to about 8. In some embodiments, the pH value of the composition is from about 6 to about 8. In some embodiments, the pH value of the composition is from about 6.5 to about 7.5. In some embodiments, the pH value of the composition is from about 4 to about 9. In some embodiments, the pH value of the composition is from about 4 to about 8. In some embodiments, the pH value of the composition is from about 5 to about 8.5. In some embodiments, the pH value of the composition is from about 6 to about 7.5.

In some embodiments, weight ratio of albumin (e.g., human serum albumin) to cabazitaxel in the composition is from about 50:1 to about 2000:1. In some embodiments, weight ratio of albumin (e.g., human serum albumin) to cabazitaxel in the composition is from about 50:1 to about 1000:1. In some embodiments, weight ratio of albumin (e.g., human serum albumin) to cabazitaxel in the composition is from about 50:1 to about 600:1. In some embodiments, weight ratio of albumin (e.g., human serum albumin) to cabazitaxel in the composition is from about 100:1 to about 2000:1. In some embodiments, weight ratio of albumin (e.g., human serum albumin) to cabazitaxel in the composition is from about 100:1 to about 1000:1. In some embodiments, weight ratio of albumin (e.g., human serum albumin) to cabazitaxel in the composition is from about 100:1 to about 600:1. In some embodiments, weight ratio of albumin (e.g., human serum albumin) to cabazitaxel in the composition is from about 50:1 to about 300:1. In some embodiments, weight ratio of albumin (e.g., human serum albumin) to cabazitaxel in the composition is from about 100:1 to about 300:1. In some embodiments, weight ratio of albumin (e.g., human serum albumin) to cabazitaxel in the composition is from about 200:1 to about 600:1. In some embodiments, weight ratio of albumin (e.g., human serum albumin) to cabazitaxel in the composition is from about 300:1 to about 800:1.

In some embodiments, the composition is a clear aqueous solution. In some embodiments, the composition stays as a clear aqueous solution for at least 1 hour, 2 hours, 4 hours, 6 hours, 8 hours, 24 hours, or 2 days. In some embodiments, the composition stays as a clear aqueous solution for at least 1 hour, 2 hours, 4 hours, 6 hours, 8 hours, 24 hours, or 2 days, when the composition keeps at from about 20° C. to about 25° C. In some embodiments, the composition stays as a clear aqueous solution for at least 1 hour, 2 hours, 4 hours, 6 hours, 8 hours, 24 hours, 2 days, or 3 days, when the composition keeps at from about 2° C. to about 8° C.

Also provided in the present invention is a composition comprising cabazitaxel and albumin, wherein the composition is an aqueous solution, wherein the concentration of Free (unbound in solution) cabazitaxel in the composition is from about 0.001 mg/ml to about 0.5 mg/ml. In some embodiments, albumin in the composition is serum albumin. In some embodiments, albumin in the composition is human serum albumin. In some embodiments, the composition is a clear aqueous solution. In some embodiments, the composition comprises a pharmaceutically acceptable vehicle (e.g. a normal saline solution or dextrose solution). In some embodiments, the composition comprises ethanol.

In some embodiments, the concentration of Free (unbound in solution) cabazitaxel in the composition is from about 0.001 mg/ml to about 0.2 mg/ml. In some embodiments, the concentration of Free (unbound in solution) cabazitaxel in the composition is from about 0.001 mg/ml to about 0.1 mg/ml. In some embodiments, the concentration of Free (unbound in solution) cabazitaxel in the composition is from about 0.002 mg/ml to about 0.2 mg/ml. In some embodiments, the concentration of Free (unbound in solution) cabazitaxel in the composition is from about 0.002 mg/ml to about 0.15 mg/ml. In some embodiments, the concentration of Free (unbound in solution) cabazitaxel in the composition is from about 0.002 mg/ml to about 0.1 mg/ml. In some embodiments, the concentration of Free (unbound in solution) cabazitaxel in the composition is from about 0.005 mg/ml to about 0.2 mg/ml. In some embodiments, the concentration of Free (unbound in solution) cabazitaxel in the composition is from about 0.005 mg/ml to about 0.15 mg/ml. In some embodiments, the concentration of Free (unbound in solution) cabazitaxel in the composition is from about 0.005 mg/ml to about 0.1 mg/ml. In some embodiments, the concentration of Free (unbound in solution) cabazitaxel in the composition is from about 0.01 mg/ml to about 0.2 mg/ml. In some embodiments, the concentration of Free (unbound in solution) cabazitaxel in the composition is from about 0.01 mg/mil to about 0.15 mg/ml. In some embodiments, the concentration of Free (unbound in solution) cabazitaxel in the composition is from about 0.01 mg/ml to about 0.1 mg/ml. In some embodiments, the concentration of Free (unbound in solution) cabazitaxel in the composition is from about 0.01 mg/ml to about 0.08 mg/ml. In some embodiments, the concentration of Free (unbound in solution) cabazitaxel in the composition is from about 0.01 mg/ml to about 0.06 mg/ml. In some embodiments, the concentration of Free (unbound in solution) cabazitaxel in the composition is from about 0.02 mg/ml to about 0.2 mg/ml. In some embodiments, the concentration of Free (unbound in solution) cabazitaxel in the composition is from about 0.02 mg/ml to about 0.15 mg/ml. In some embodiments, the concentration of Free (unbound in solution) cabazitaxel in the composition is from about 0.02 mg/ml to about 0.1 mg/ml. In some embodiments, the concentration of Free (unbound in solution) cabazitaxel in the composition is from about 0.02 mg/ml to about 0.08 mg/ml. In some embodiments, the concentration of Free (unbound in solution) cabazitaxel in the composition is from about 0.02 mg/ml to about 0.06 mg/ml. In some embodiments, the concentration of Free (unbound in solution) cabazitaxel in the composition is from about 0.03 mg/ml to about 0.1 mg/ml. In some embodiments, the concentration of Free (unbound in solution) cabazitaxel in the composition is from about 0.03 mg/ml to about 0.08 mg/ml. In some embodiments, the concentration of Free (unbound in solution) cabazitaxel in the composition is from about 0.03 mg/ml to about 0.06 mg/ml. In some embodiments, the concentration of Free (unbound in solution) cabazitaxel in the composition is from about 0.025 mg/ml to about 0.075 mg/ml. In some embodiments, the concentration of Free (unbound in solution) cabazitaxel in the composition is from about 0.025 mg/ml to about 0.055 mg/ml. In some embodiments, the concentration of Free (unbound in solution) cabazitaxel in the composition is from about 0.04 mg/ml to about 0.06 mg/ml. In some embodiments, the concentration of Free (unbound in solution) cabazitaxel in the composition is about 0.010 mg/ml, about 0.015 mg/ml, about 0.020 mg/ml, about 0.025 mg/ml, about 0.030 mg/ml, about 0.035 mg/ml, about 0.040 mg/ml, about 0.045 mg/ml, about 0.050 mg/ml, about 0.055 mg/ml, about 0.060 mg/ml, about 0.065 mg/ml, about 0.070 mg/ml, about 0.075 mg/ml, about 0.080 mg/ml, about 0.085 mg/ml, about 0.090 mg/ml, about 0.095 mg/ml, or about 0.10 mg/ml.

Fraction of free (unbound in solution) cabazitaxel in the composition is measured by ultrafiltration through a 30-kDa membrane. The experiments of free (unbound in solution) cabazitaxel measurement are described in the example section.

In some embodiments, the concentration of albumin (e.g., human serum albumin) in the composition is from about 0.1% to about 25% (w/v). In some embodiments, the concentration of albumin (e.g., human serum albumin) in the composition is from about 0.5% to about 20% (w/v). In some embodiments, the concentration of albumin (e.g., human serum albumin) in the composition is from about 0.5% to about 10% (w/v). In some embodiments, the concentration of albumin (e.g., human serum albumin) in the composition is from about 1% to about 10% (w/v). In some embodiments, the concentration of albumin (e.g., human serum albumin) in the composition is from about 0.5% to about 5% (w/v). In some embodiments, the concentration of albumin (e.g., human serum albumin) in the composition is from about 5% to about 10% (w/v). In some embodiments, the concentration of albumin (e.g., human serum albumin) in the composition is from about 1% to about 5% (w/v). In some embodiments, the concentration of albumin (e.g., human serum albumin) in the composition is from about 0.5% to about 3% (w/v). In some embodiments, the concentration of albumin (e.g., human serum albumin) in the composition is from about 0.3% to about 3% (w/v). In some embodiments, the concentration of albumin (e.g., human serum albumin) in the composition is from about 0.2% to about 6% (w/v). In some embodiments, the concentration of albumin (e.g., human serum albumin) in the composition is from about 2% to about 6% (w/v).

In some embodiments, the composition comprises ethanol. In some embodiments, the amount of ethanol in the composition is no more than 15% (v/v). In some embodiments, the amount of ethanol in the composition is no more than 10% (v/v). In some embodiments, the amount of ethanol in the composition is no more than 5% (v/v). In some embodiments, the amount of ethanol in the composition is no more than 3% (v/v). In some embodiments, the amount of ethanol in the composition is from about 0.1% to about 15% (v/v). In some embodiments, the amount of ethanol in the composition is from about 0.2% to about 10% (v/v). In some embodiments, the amount of ethanol in the composition is from about 0.10% to about 10% (v/v). In some embodiments, the amount of ethanol in the composition is from about 0.5% to about 3% (v/v). In some embodiments, the amount of ethanol in the composition is from about 0.1% to about 3% (v/v). In some embodiments, the amount of ethanol in the composition is from about 1% to about 5% (v/v). In some embodiments, the amount of ethanol in the composition is from about 0.1% to about 5% (v/v). In some embodiments, the amount of ethanol in the composition is from about 0.5% to about 5% (v/v). In some embodiments, the amount of ethanol in the composition is from about 0.5% to about 10% (v/v). In some embodiments, the amount of ethanol in the composition is from about 2% to about 8% (v/v).

In some embodiments, the composition comprises pH adjusting agent(s). Suitable pH adjusting agents include, but are not limited to, diethanolamine, triethanolamine, sodium hydroxide, hydrochloric acid, citric acid and monobasic sodium phosphate and the like or their mixtures thereof. In some embodiments, pH adjusting agent is citric acid. In some embodiments, the pH value of the composition is from about 4 to about 9.5. In some embodiments, the pH value of the composition is from about 5 to about 9. In some embodiments, the pH value of the composition is from about 5 to about 8. In some embodiments, the pH value of the composition is from about 6 to about 8. In some embodiments, the pH value of the composition is from about 6.5 to about 7.5. In some embodiments, the pH value of the composition is from about 4 to about 9. In some embodiments, the pH value of the composition is from about 4 to about 8. In some embodiments, the pH value of the composition is from about 5 to about 8.5. In some embodiments, the pH value of the composition is from about 6 to about 7.5.

In some embodiments, weight ratio of albumin (e.g., human serum albumin) to cabazitaxel in the composition is from about 50:1 to about 2000:1. In some embodiments, weight ratio of albumin (e.g., human serum albumin) to cabazitaxel in the composition is from about 50:1 to about 1000:1. In some embodiments, weight ratio of albumin (e.g., human serum albumin) to cabazitaxel in the composition is from about 50:1 to about 600:1. In some embodiments, weight ratio of albumin (e.g., human serum albumin) to cabazitaxel in the composition is from about 100:1 to about 2000:1. In some embodiments, weight ratio of albumin (e.g., human serum albumin) to cabazitaxel in the composition is from about 100:1 to about 1000:1. In some embodiments, weight ratio of albumin (e.g., human serum albumin) to cabazitaxel in the composition is from about 100:1 to about 600:1. In some embodiments, weight ratio of albumin (e.g., human serum albumin) to cabazitaxel in the composition is from about 50:1 to about 300:1. In some embodiments, weight ratio of albumin (e.g., human serum albumin) to cabazitaxel in the composition is from about 100:1 to about 300:1. In some embodiments, weight ratio of albumin (e.g., human serum albumin) to cabazitaxel in the composition is from about 200:1 to about 600:1. In some embodiments, weight ratio of albumin (e.g., human serum albumin) to cabazitaxel in the composition is from about 300:1 to about 800:1.

In some embodiments, the composition is a clear aqueous solution. In some embodiments, the composition stays as a clear aqueous solution for at least 1 hour, 2 hours, 4 hours, 6 hours, 8 hours, 24 hours, or 2 days. In some embodiments, the composition stays as a clear aqueous solution for at least 1 hour, 2 hours, 4 hours, 6 hours, 8 hours, 24 hours, or 2 days, when the composition keeps at from about 20° C. to about 25° C. In some embodiments, the composition stays as a clear aqueous solution for at least 1 hour, 2 hours, 4 hours, 6 hours, 8 hours, 24 hours, 2 days, or 3 days, when the composition keeps at from about 2° C. to about 8° C.

Also, provided herein is an injectable pharmaceutical composition comprising cabazitaxel and ethanol, wherein the said injectable pharmaceutical composition comprises from about 5 mg to 200 mg of cabazitaxel, and wherein the said injectable pharmaceutical composition is further mixed with an aqueous composition comprising human serum albumin to form a parenteral infusion solution prior to being infused or administered to patients. In some embodiments, the injectable pharmaceutical composition does not contain Polysorbate 80. In some embodiments, the injectable pharmaceutical composition further comprises citric acid. In some embodiments, the mixing of the injectable pharmaceutical composition with the aqueous composition comprising human serum albumin is done in an infusion bag or bottle. In some embodiments, the parenteral infusion solution obtained after mixing in an infusion bag or bottle is a transparent infusion solution free of precipitates.

In some embodiments, the mixing of the injectable pharmaceutical composition with the aqueous composition comprising human serum albumin includes two steps. In the first step, the injectable pharmaceutical composition is injected into the infusion bag or bottle containing the aqueous composition comprising human serum albumin. In the injection process, the infusion bag or bottle is held still and mixing or agitation is not need. In the second step, after the injection of the injectable pharmaceutical composition into the infusion bag or bottle is completed, the injectable pharmaceutical composition and the aqueous composition are mixed well (e.g., the infusion bag or bottle containing the composition is gently inverted by hand) to obtain a parenteral infusion solution.

In some embodiments, provided herein is a cabazitaxel injection free of Polysorbate 80 comprising cabazitaxel and ethanol. In some embodiments, provided herein is a cabazitaxel injection free of Polysorbate 80 comprising cabazitaxel, citric acid, and ethanol. In some embodiments, provided herein is a cabazitaxel injection free of Polysorbate 80 comprising cabazitaxel, citric acid, PEG300, and ethanol. In some embodiments, provided herein is a cabazitaxel injection free of Polysorbate 80 consisting essentially of cabazitaxel, citric acid, and ethanol. In some embodiments, provided herein is a cabazitaxel injection free of Polysorbate 80 consisting essentially of cabazitaxel, citric acid, PEG300, and ethanol.

In some embodiments, the injectable pharmaceutical composition comprises from about 5 mg to about 150 mg of cabazitaxel. In some embodiments, the injectable pharmaceutical composition comprises from about 5 mg to about 100 mg of cabazitaxel. In some embodiments, the injectable pharmaceutical composition comprises from about 10 mg to about 80 mg of cabazitaxel. In some embodiments, the injectable pharmaceutical composition comprises from about 10 mg to about 60 mg of cabazitaxel. In some embodiments, the injectable pharmaceutical composition comprises 5 mg, 10 mg, 15 mg, 20 mg, 25 mg, 30 mg, 35 mg, 40 mg, 45 mg, 50 mg, 55 mg, or 60 mg of cabazitaxel. In some embodiments, the injectable pharmaceutical composition comprises about 5 mg, about 10 mg, about 15 mg, about 20 mg, about 25 mg, about 30 mg, about 35 mg, about 40 mg, about 45 mg, about 50 mg, about 55 mg, or about 60 mg of cabazitaxel.

In some embodiments, the injectable pharmaceutical composition does not comprise a lipid (e.g. soybean oil). In some embodiments, the injectable pharmaceutical composition does not comprise a surfactant. In some embodiments, the injectable pharmaceutical composition contains one or more other organic solvents (e.g. propylene glycol, polyethylene glycol 300, polyethylene glycol 400, etc.) in addition to ethanol. In some embodiments, the injectable pharmaceutical composition contains polyethylene glycol 300 (PEG300). In some embodiments, the injectable pharmaceutical composition contains more than 50% ethanol (v/v). In some embodiments, the injectable pharmaceutical composition contains more than 70% ethanol (v/v). In some embodiments, the injectable pharmaceutical composition contains more than 80% ethanol (v/v).

In some embodiments, the concentration of cabazitaxel in the injectable pharmaceutical composition is from about 1 mg/ml to about 40 mg/ml. In some embodiments, the concentration of cabazitaxel in the injectable pharmaceutical composition is from about 5 mg/ml to about 20 mg/ml. In some embodiments, the concentration of cabazitaxel in the injectable pharmaceutical composition is from about 5 mg/ml to about 15 mg/ml. In some embodiments, the concentration of cabazitaxel in the injectable pharmaceutical composition is about 5 mg/ml, about 10 mg/ml, about 15 mg/ml or about 20 mg/ml.

In some embodiments, the injectable pharmaceutical composition further comprises an acid. In some embodiments, the acid is an organic acid. The most preferred acid for use in accordance with the present invention is citric acid.

In some embodiments, pH value of the injectable pharmaceutical composition is from about 3 to about 7. In some embodiments, pH value of the injectable pharmaceutical composition is from about 3 to about 6.5. In some embodiments, pH value of the injectable pharmaceutical composition is from about 3.5 to about 6.5. In some embodiments, pH value of the injectable pharmaceutical composition is from about 3.5 to about 6. In some embodiments, pH value of the injectable pharmaceutical composition is from about 3.5 to about 5.5. In some embodiments, pH value of the injectable pharmaceutical composition is from about 4 to about 6. To measure the pH value of the injectable pharmaceutical composition, the injectable pharmaceutical composition is mixed with water in 3:2 ratio (v/v) to obtain an aqueous solution, and then pH value of the aqueous solution is tested.

According to yet another embodiment, the injectable pharmaceutical composition of cabazitaxel is formulated into an injectable pharmaceutical dosage form. In some embodiments, the injectable pharmaceutical dosage form comprises from about 10 mg to about 100 mg of cabazitaxel. In some embodiments, the injectable pharmaceutical dosage form comprises from about 10 mg to about 60 mg of cabazitaxel. In some embodiments, the injectable pharmaceutical dosage form comprises about 10 mg, about 20 mg, about 30 mg, about 40 mg, about 50 mg, or about 60 mg of cabazitaxel. In some embodiments, the concentration of cabazitaxel in the injectable pharmaceutical dosage form is from 1 mg/ml to 30 mg/ml. In some embodiments, the concentration of cabazitaxel in the injectable pharmaceutical dosage form is from 5 mg/ml to 20 mg/ml. In some embodiments, the injectable pharmaceutical dosage form comprises about 10 mg, about 20 mg, about 30 mg, about 40 mg, about 50 mg, or about 60 mg of cabazitaxel, citric acid and ethanol, wherein the concentration of cabazitaxel in the injectable pharmaceutical dosage form is from 5 mg/ml to 20 mg/ml (e.g., about 10 mg/ml, about 20 mg/ml). In some embodiments, the injectable pharmaceutical dosage form has pH value from about 3 to about 6.5. The preferred pH of the injectable pharmaceutical dosage form is from about 3.5 to about 6.0.

Also, provided herein is a method of treating cancer, the method comprising administering to a subject in need thereof a therapeutically effective amount of any composition or formulation described herein. In some embodiments, the method comprises parenterally administering to a subject in need thereof of a therapeutically effective amount of a liquid infusion composition of the present disclosure.

In some embodiments, the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

In some embodiments, cancer is selected from sarcoma, angiosarcoma, fibrosarcoma, rhabdomyosarcoma, liposarcoma, myxoma, rhabdomyoma, fibroma, lipoma, teratoma, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the cancer is selected from breast cancer, non-small cell lung cancer, hormone refractory prostate cancer, gastric adenocarcinoma, and squamous cell carcinoma of head and neck cancer.

### EXAMPLES

### Example 1

50 mg of cabazitaxel was dissolved in 5 ml of dehydrated ethanol to obtain a cabazitaxel API solution. 10 ml of a 20% solution of human serum albumin for infusion (w/v) was added into a 250 ml infusion bottle containing 188 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 25 times, and the solution was mixed well to obtain a transparent solution free of visible particles or precipitates. The particulate matter of the transparent solution was also measured. 1 ml of the transparent solution has about 49 particles that are greater than 10 μm in size and about 11 particles that are greater than 25 μm in size. The solution was kept at 20° C. to 25° C. for 6 hours, which stayed as a transparent solution free of visible particles or precipitates for 6 hours. The particulate matter of the transparent solution was also measured at 6 hours. 1 ml of the transparent solution at 6 hour has about 49 particles that are greater than 10 μm in size and about 5 particles that are greater than 25 μm in size.

12 ml of a 20% solution of human serum albumin for infusion (w/v) was added into a 250 ml infusion bottle containing 186 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 25 times, and the solution was mixed well to obtain a transparent solution free of visible particles or precipitates. The particulate matter of the transparent solution was also measured. 1 ml of the transparent solution has about 55 particles that are greater than 10 μm in size and about 4 particles that are greater than 25 μm in size. The solution was kept at 20° C. to 25° C. for 6 hours, which stayed as a transparent solution free of visible particles or precipitates for 6 hours. The particulate matter of the transparent solution was also measured at 6 hour. 1 ml of the transparent solution at 6 hour has about 31 particles that are greater than 10 μm in size and about 0 particles that are greater than 25 μm in size.

### Example 2

20.4 mg citric acid was dissolved in 20 ml dehydrated ethanol to obtain a citric acid solution (about 1 mg/ml). 50.2 mg of cabazitaxel was dissolved in 4.9 ml of dehydrated ethanol, followed by adding 0.1 ml of the prepared citric acid solution to obtain a cabazitaxel API solution. 8 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 25 times, and the solution was mixed well to obtain a transparent solution free of visible particles or precipitates. The particulate matter of the transparent solution was also measured. 1 ml of the transparent solution has about 23 particles that are greater than 10 μm in size and 0 particle that are greater than 25 μm in size. The solution was kept at 20° C. to 25° C. for 6 hours, which stayed as a transparent solution free of visible particles or precipitates for 6 hours. The particulate matter of the transparent solution was also measured at 6 hour. 1 ml of the transparent solution at 6 hour has about 85 particles that are greater than 10 μm in size and about 2 particles that are greater than 25 μm in size.

6 ml of a 20% solution of human serum albumin for infusion (w/v) was added into a 250 ml infusion bottle containing 192 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 25 times, and the solution was mixed well to obtain a transparent solution free of visible particles or precipitates. The particulate matter of the transparent solution was also measured. 1 ml of the transparent solution has about 130 particles that are greater than m in size and about 9 particles that are greater than 25 μm in size. The solution was kept at 20° C. to 25° C. for 6 hours, which stayed as a transparent solution free of visible particles or precipitates for 6 hours. The particulate matter of the transparent solution was also measured at 6 hour. 1 ml of the transparent solution at 6 hour has about 62 particles that are greater than 10 μm in size and 0 particle that are greater than 3 μm in size.

### Example 3

20.41 mg citric acid was dissolved in 20 ml dehydrated ethanol to obtain a citric acid solution (about 1 mg/ml). 50.39 mg of cabazitaxel was dissolved in 4.9 ml of dehydrated ethanol, followed by adding 0.1 ml of the prepared citric acid solution to obtain a cabazitaxel API solution. 5 ml of a 20% solution of human serum albumin for infusion (w/v) was added into a 250 ml infusion bottle containing 193 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 25 times, and the solution was mixed well to obtain a transparent solution free of visible particles or precipitates. The particulate matter of the transparent solution was also measured. 1 ml of the transparent solution has about 131 particles that are greater than 10 μm in size and about 2 particles that are greater than 25 μm in size. The solution was kept at 20° C. to 25° C. for 5 hours, which stayed as a transparent solution free of visible particles or precipitates for 5 hours. The particulate matter of the transparent solution was also measured at 5 hour. 1 ml of the transparent solution at 5 hour has about 53 particles that are greater than 10 μm in size and about 3 particles that are greater than 25 μm in size.

### Example 4

8.79 mg citric acid was dissolved in 8.8 ml dehydrated ethanol to obtain a citric acid solution (about 1 mg/ml). 50.02 mg of cabazitaxel was dissolved in 4.9 ml of dehydrated ethanol, followed by adding 0.1 ml of the prepared citric acid solution to obtain a cabazitaxel API solution. 10 ml of a 20% solution of human serum albumin for infusion (w/v) was added into a 500 ml infusion bottle containing 238 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 15 times, and the solution was mixed well to obtain a transparent solution free of visible particles or precipitates. The particulate matter of the transparent solution was also measured. 1 ml of the transparent solution has about 41 particles that are greater than 10 μm in size and 0 particle that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 61 particles that are greater than 10 μm in size and about 1 particles that are greater than 25 μm in size.

### Example 5

5.9 mg citric acid was dissolved in 5.9 ml dehydrated ethanol to obtain a citric acid solution (about 1 mg/ml). 50.1 mg of cabazitaxel was dissolved in 4.9 ml of dehydrated ethanol, followed by adding 0.1 ml of the prepared citric acid solution to obtain a cabazitaxel API solution. 8 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 1.6 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter of the transparent solution was also measured. 1 ml of the transparent solution has about 84 particles that are greater than 10 μm in size and about 7 particles that are greater than 25 μm in size. The solution was kept at 20° C. to 25° C. for 5 hours, which stayed as a transparent solution free of visible particles or precipitates for 5 hours. The particulate matter of the transparent solution was also measured at 5 hour. 1 ml of the transparent solution at 5 hour has about 162 particles that are greater than 10 μm in size and about 9 particles that are greater than 25 μm in size.

### Example 6

14.3 mg citric acid was dissolved in 14.3 ml dehydrated ethanol to obtain a citric acid solution (about 1 mg/ml). 200.7 mg of cabazitaxel was dissolved in 19.6 ml of dehydrated ethanol, followed by adding 0.4 ml of the prepared citric acid solution to obtain a cabazitaxel API solution. 20 ml of a 20% solution of human serum albumin for infusion (w/v) was added into a 500 ml infusion bottle containing 376 ml normal saline solution (0.9% NaCl solution). After the albumin solution was mixed well with saline solution, the infusion bottle was kept still at horizontal position, and 4 ml of the cabazitaxel API solution was injected rapidly into the infusion bottle by injecting underneath the liquid surface of the albumin solution (the injection process took about 4 seconds). Then the infusion bottle was immediately gently inverted for about 20 times (the inversion process took about 50 seconds), and the solution was mixed well to obtain a transparent solution free of visible particles or precipitates. The particulate matter of the transparent solution was also measured. 1 ml of the transparent solution has about 40 particles that are greater than 10 μm in size and about 3 particles that are greater than 25 μm in size. The solution was kept at 20° C. to 25° C. for 8 hours, which stayed as a transparent solution free of visible particles or precipitates for 8 hours. The particulate matter of the transparent solution was also measured at 8 hour. 1 ml of the transparent solution at 8 hour has about 17 particles that are greater than 10 μm in size and about 0 particles that are greater than 25 μm in size.

### Example 7

14.3 mg citric acid was dissolved in 14.3 ml dehydrated ethanol to obtain a citric acid solution (about 1 mg/ml). 200.7 mg of cabazitaxel was dissolved in 19.6 ml of dehydrated ethanol, followed by adding 0.4 ml of the prepared citric acid solution to obtain a cabazitaxel API solution. 15 ml of a 20% solution of human serum albumin for infusion (w/v) was added into a 500 ml infusion bottle containing 380 ml normal saline solution (0.9% NaCl solution). After the albumin solution was mixed well with saline solution, the infusion bottle was kept still at horizontal position, and 3 ml of the cabazitaxel API solution was injected rapidly into the infusion bottle by injecting underneath the liquid surface of the albumin solution (the injection process took about 3 seconds). Then the infusion bottle was immediately gently inverted for about 20 times (the inversion process took about 50 seconds), and the solution was mixed well to obtain a transparent solution free of visible particles or precipitates. The particulate matter of the transparent solution was also measured. 1 ml of the transparent solution has about 6 particles that are greater than 10 μm in size and 0 particle that are greater than 25 μm in size. The solution was kept at 20° C. to 25° C. for 8 hours, which stayed as a transparent solution free of visible particles or precipitates for 8 hours. The particulate matter of the transparent solution was also measured at 8 hour. 1 ml of the transparent solution at 8 hour has about 23 particles that are greater than 10 μm in size and about 1 particle that are greater than 25 μm in size.

22 ml of a 20% solution of human serum albumin for infusion (w/v) was added into a 500 ml infusion bottle containing 380 ml normal saline solution (0.9% NaCl solution). After the albumin solution was mixed well with saline solution, the infusion bottle was kept still at horizontal position, and 4.4 ml of the cabazitaxel API solution was injected rapidly into the infusion bottle by injecting underneath the liquid surface of the albumin solution (the injection process took about 4-5 seconds). Then the infusion bottle was immediately gently inverted for about 20 times (the inversion process took about 50 seconds), and the solution was mixed well to obtain a transparent solution free of visible particles or precipitates. The particulate matter of the transparent solution was also measured. 1 ml of the transparent solution has about 29 particles that are greater than 10 μm in size and 0 particle that are greater than 25 μm in size. The solution was kept at 20° C. to 25° C. for 8 hours, which stayed as a transparent solution free of visible particles or precipitates for 8 hours. The particulate matter of the transparent solution was also measured at 8 hour. 1 ml of the transparent solution at 8 hour has about 40 particles that are greater than 10 μm in size and about 4 particles that are greater than 25 μm in size.

### Example 8

10.4 mg citric acid was dissolved in 10.4 ml dehydrated ethanol to obtain a citric acid solution (about 1 mg/ml). 200.3 mg of cabazitaxel was dissolved in 19.6 ml of dehydrated ethanol, followed by adding 0.4 ml of the prepared citric acid solution to obtain a cabazitaxel API solution. 7.5 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 1.5 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times (the inversion process took about 50 seconds), and the solution was mixed well to obtain a transparent solution free of visible particles or precipitates. The particulate matter of the transparent solution was also measured. 1 ml of the transparent solution has about 16 particles that are greater than 10 μm in size and about 1 particle that are greater than 25 μm in size. The solution was kept at 20° C. to 25° C. for 6 hours, which stayed as a transparent solution free of visible particles or precipitates for 6 hours. The particulate matter of the transparent solution was also measured at 6 hour. 1 ml of the transparent solution at 6 hour has about 16 particles that are greater than 10 μm in size and about 1 particle that are greater than 25 μm in size.

10 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 2 ml of the cabazitaxel API solution was injected rapidly into the infusion bottle by injecting underneath the liquid surface of the albumin solution (the injection process took about 2 seconds). Then the infusion bottle was immediately gently inverted for about 20 times (the inversion process took about 50 seconds), and the solution was mixed well to obtain a transparent solution free of visible particles or precipitates. The particulate matter of the transparent solution was also measured. 1 ml of the transparent solution has about 81 particles that are greater than 10 μm in size and about 6 particles that are greater than 25 μm in size. The solution was kept at 20° C. to 25° C. for 6 hours, which stayed as a transparent solution free of visible particles or precipitates for 6 hours. The particulate matter of the transparent solution was also measured at 6 hour. 1 ml of the transparent solution at 6 hour has about 43 particles that are greater than 10 μm in size and 0 particle that are greater than 25 μm in size.

11 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 2.2 ml of the cabazitaxel API solution was injected rapidly into the infusion bottle by injecting underneath the liquid surface of the albumin solution (the injection process took about 2 seconds). Then the infusion bottle was immediately gently inverted for about 20 times (the inversion process took about 50 seconds), and the solution was mixed well to obtain a transparent solution free of visible particles or precipitates. The particulate matter of the transparent solution was also measured. 1 ml of the transparent solution has about 34 particles that are greater than 10 μm in size and about 1 particle that are greater than 25 μm in size. The solution was kept at 20° C. to 25° C. for 6 hours, which stayed as a transparent solution free of visible particles or precipitates for 6 hours. The particulate matter of the transparent solution was also measured at 6 hour. 1 ml of the transparent solution at 6 hour has about 53 particles that are greater than 10 μm in size and about 2 particles that are greater than 25 μm in size.

### Example 9

13.1 mg citric acid was dissolved in 13.1 ml dehydrated ethanol to obtain a citric acid solution (about 1 mg/ml). 199.7 mg of cabazitaxel was dissolved in 19.6 ml of dehydrated ethanol, followed by adding 0.4 ml of the prepared citric acid solution to obtain a cabazitaxel API solution. 20 ml of a 20% solution of human serum albumin for infusion (w/v) was added into a 500 ml infusion bottle containing 476 ml normal saline solution (0.9% NaCl solution). After the albumin solution was mixed well with saline solution, the infusion bottle was kept still at horizontal position, and 4 ml of the cabazitaxel API solution was injected rapidly into the infusion bottle by injecting underneath the liquid surface of the albumin solution (the injection process took about 4 seconds). Then the infusion bottle was immediately gently inverted for about 30 times (the inversion process took about 80 seconds), and the solution was mixed well to obtain a transparent solution free of visible particles or precipitates. The solution was kept at 20° C. to 25° C. for 8 hours, which stayed as a transparent solution free of visible particles or precipitates for 8 hours.

12 ml of a 20% solution of human serum albumin for infusion (w/v) was added into a 500 ml infusion bottle containing 285 ml normal saline solution (0.9% NaCl solution). After the albumin solution was mixed well with saline solution, the infusion bottle was kept still at horizontal position, and 2.4 ml of the cabazitaxel API solution was injected rapidly into the infusion bottle by injecting underneath the liquid surface of the albumin solution (the injection process took about 2.5 seconds). Then the infusion bottle was immediately gently inverted for about 20 times (the inversion process took about 60 seconds), and the solution was mixed well to obtain a transparent solution free of visible particles or precipitates. The solution was kept at 20° C. to 25° C. for 4 hours, which stayed as a transparent solution free of visible particles or precipitates for 4 hours.

### Example 10

12 mg citric acid was dissolved in 12 ml dehydrated ethanol to obtain a citric acid solution (about 1 mg/ml). 100.2 mg of cabazitaxel was dissolved in 9.8 ml of dehydrated ethanol, followed by adding 0.2 ml of the prepared citric acid solution to obtain a cabazitaxel API solution. 7 ml of a 20% solution of human serum albumin for infusion (w/v) was added into a 250 ml infusion bottle containing 191 ml normal saline solution (0.9% NaCl solution). After the albumin solution was mixed well with saline solution, the infusion bottle was kept still at horizontal position, and 1.4 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter of the transparent solution was also measured. 1 ml of the transparent solution has about 129 particles that are greater than 10 μm in size and about 8 particles that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 65 particles that are greater than 10 μm in size and 0 particle that are greater than 25 μm in size.

8 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 1.6 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter of the transparent solution was also measured. 1 ml of the transparent solution has about 71 particles that are greater than 10 μm in size and about 5 particles that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 89 particles that are greater than 10 μm in size and about 1 particle that are greater than 25 μm in size.

9 ml of a 20% solution of human serum albumin for infusion (w/v) was added into a 250 ml infusion bottle containing 189 ml normal saline solution (0.9% NaCl solution). After the albumin solution was mixed well with saline solution, the infusion bottle was kept still at horizontal position, and 1.8 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter of the transparent solution was also measured. 1 ml of the transparent solution has about 38 particles that are greater than 10 μm in size and about 10 particles that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 113 particles that are greater than 10 μm in size and about 3 particles that are greater than 25 μm in size.

### Example 11

13.5 mg citric acid was dissolved in 13.5 ml dehydrated ethanol to obtain a citric acid solution (about 1 mg/ml). 100 mg of cabazitaxel was dissolved in 9.8 ml of dehydrated ethanol, followed by adding 0.2 ml of the prepared citric acid solution to obtain a cabazitaxel API solution-A. 8 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 1.6 ml of the cabazitaxel API solution-A was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter and pH of the transparent solution were also measured. 1 ml of the transparent solution has about 31 particles that are greater than 10 μm in size and about 13 particles that are greater than 25 μm in size. The pH of the transparent solution is 6.82. The solution was divided into two portions. One portion of the solution was kept at 20° C. to 25° C. for 8 hours, and the other portion of the solution was kept at 2° C. to 8° C. for 8 hours. Both solutions stayed as transparent solutions free of visible particles or precipitates for 8 hours.

13.5 mg citric acid was dissolved in 13.5 ml dehydrated ethanol to obtain a citric acid solution (about 1 mg/ml). 100.1 mg of cabazitaxel was dissolved in 7.8 ml of dehydrated ethanol, followed by adding 0.2 ml of the prepared citric acid solution and 2 ml of PEG 300 to obtain a cabazitaxel API solution-B. 8 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 1.6 ml of the cabazitaxel API solution-B was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter and pH of the transparent solution were also measured. 1 ml of the transparent solution has about 27 particles that are greater than 10 μm in size and about 11 particles that are greater than 25 μm in size. The pH of the transparent solution is 6.79. The solution was divided into two portions. One portion of the solution was kept at 20° C. to 25° C. for 8 hours, and the other portion of the solution was kept at 2° C. to 8° C. for 8 hours. Both solutions stayed as transparent solutions free of visible particles or precipitates for 8 hours.

13.5 mg citric acid was dissolved in 13.5 ml dehydrated ethanol to obtain a citric acid solution (about 1 mg/ml). 99.8 mg of cabazitaxel was dissolved in 7.8 ml of dehydrated ethanol, followed by adding 0.2 ml of the prepared citric acid solution and 2 ml of propylene glycol to obtain a cabazitaxel API solution-C. 8 ml of a 20% solution of human serum albumin for infusion (w/v) was added into a 250 ml infusion bottle containing 190 ml normal saline solution (0.9% NaCl solution). After the albumin solution was mixed well with saline solution, the infusion bottle was kept still at horizontal position, and 1.6 ml of the cabazitaxel API solution-C was injected rapidly into the infusion bottle by injecting underneath the liquid surface of the albumin solution (the injection process took about 1-2 seconds). Then the infusion bottle was immediately gently inverted for about 20 times, and the solution was mixed well to obtain a transparent solution free of visible particles or precipitates. The particulate matter and pH of the transparent solution were also measured. 1 ml of the transparent solution has about 173 particles that are greater than 10 μm in size and about 12 particles that are greater than 25 μm in size. The pH of the transparent solution is 6.84. The solution was divided into two portions. One portion of the solution was kept at 20° C. to 25° C. for 7 hours, and the other portion of the solution was kept at 2° C. to 8° C. for 7 hours. Both solutions stayed as a transparent solution free of visible particles or precipitates for 7 hours.

### Example 12

17.5 mg citric acid was dissolved in 17.5 ml dehydrated ethanol to obtain a citric acid solution (about 1 mg/ml). 100.5 mg of cabazitaxel was dissolved in 9.8 ml of dehydrated ethanol, followed by adding 0.2 ml of the prepared citric acid solution to obtain a cabazitaxel API solution. 8 ml of a 20% solution of human serum albumin for infusion (w/v) was added into a 250 ml infusion bottle containing 190 ml 5% dextrose solution. After the albumin solution was mixed well with dextrose solution, the infusion bottle was kept still at horizontal position, and 1.6 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times (the inversion process took about 60 seconds), and the solution was mixed well to obtain a transparent solution free of visible particles or precipitates. The particulate matter of the transparent solution was also measured. 1 ml of the transparent solution has about 68 particles that are greater than 10 μm in size and about 6 particles that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 57 particles that are greater than 10 μm in size and about 6 particles that are greater than 25 μm in size.

### Example 13

29.5 mg citric acid was dissolved in 29.5 ml dehydrated ethanol to obtain a citric acid solution (about lmg/ml). 59.9 mg of cabazitaxel was dissolved in 6 ml of dehydrated ethanol to obtain a Formular A cabazitaxel product. 60.1 mg of cabazitaxel was dissolved in 5.76 ml of dehydrated ethanol, followed by adding 0.24 ml of the prepared citric acid solution to obtain a Formular B cabazitaxel product. 60.6 mg of cabazitaxel was dissolved in 5.88 ml of dehydrated ethanol, followed by adding 0.12 ml of the prepared citric acid solution to obtain a Formular C cabazitaxel product. 60.9 mg of cabazitaxel was dissolved in 5.97 ml of dehydrated ethanol, followed by adding 0.03 ml of the prepared citric acid solution to obtain a Formular D cabazitaxel product. 59.4 mg of cabazitaxel was dissolved in 5.988 ml of dehydrated ethanol, followed by adding 0.012 ml ofthe prepared citric acid solution to obtain a Formular E cabazitaxel product. All 5 cabazitaxel products were studied for stability tests at 2-8° C., 25° C., and 40′° C. The data of the stability studies have indicated that adding citric acid into the cabazitaxel formulations significantly increases the stability of cabazitaxel in the formulations, of which Formular B and Formular C are most stable formulations.

TABLE 1
Stability Test of 5 cabazitaxel products at 40° C.
(Total Impurity Amount)

| | Total Impurity Amount | | | | |
|---|---|---|---|---|---|
| | Formu- | Formu- | Formu- | Formu- | Formu- |
| | lar A | lar B | lar C | lar D | lar E |
| Ratio (citric | 0 | 1:250 | 1:500 | 1:2000 | 1:5000 |
| acid:cabazitaxel) | | | | | |
| 0 day | 0.15% | 0.08% | 0.078% | 0.086% | 0.096% |
| 1 day | 2.866% | 0.055% | 0.058% | 0.258% | 0.959% |
| 4 days | 8.775% | 0.082% | 0.076% | 0.974% | 3.759% |
| 6 days | 12.285% | 0.105% | 0.086% | 1.639% | 5.773% |
| 8 days | 15.176% | 0.137% | 0.128% | 2.357% | 7.716% |
| 11 days | NT | 0.153% | 0.157% | NT | NT |

TABLE 2
Stability Test of 5 cabazitaxel products at 25° C.
(Total Impurity Amount)

| | Total Impurity Amount | | | | |
|---|---|---|---|---|---|
| | Formu- | Formu- | Formu- | Formu- | Formu- |
| | lar A | lar B | lar C | lar D | lar E |
| Ratio (citric | 0 | 1:250 | 1:500 | 1:2000 | 1:5000 |
| acid:cabazitaxel) | | | | | |
| 0 day | 0.15% | 0.08% | 0.078% | 0.086% | 0.096% |
| 1 day | 0.371% | 0.048% | 0.051% | 0.125% | 0.148% |
| 4 days | 1.173% | 0.071% | 0.068% | 0.152% | 0.373% |
| 8 days | 2.329% | 0.105% | 0.098% | 0.341% | 0.764% |
| 11 days | 3.254% | 0.132% | 0.122% | 0.425% | 1.139% |

TABLE 3
Stability Test of 5 cabazitaxel products at 2-8° C.
(Total Impurity Amount)

| | Total Impurity Amount | | | | |
|---|---|---|---|---|---|
| | Formu- | Formu- | Formu- | Formu- | Formu- |
| | lar A | lar B | lar C | lar D | lar E |
| Ratio (citric | 0 | 1:250 | 1:500 | 1:2000 | 1:5000 |
| acid:cabazitaxel) | | | | | |
| 0 day | 0.15% | 0.08% | 0.078% | 0.086% | 0.096% |
| 1 day | 0.122% | 0.058% | 0.062% | 0.061% | 0.081% |
| 4 days | 0.202% | 0.072% | 0.071% | 0.094% | 0.122% |
| 11 days | 0.413% | 0.128% | 0.135% | 0.142% | 1.0% |

### Example 14

8.4 mg citric acid was dissolved in 8.4 ml dehydrated ethanol to obtain a citric acid solution (about 1 mg/ml). 299.2 mg of cabazitaxel was dissolved in 29.4 ml of dehydrated ethanol, followed by adding 0.6 ml of the prepared citric acid solution to obtain a cabazitaxel API solution. 16 ml of a 20% solution of human serum albumin for infusion (w/v) was added into a 250 ml infusion bottle containing 83 ml normal saline solution (0.9% NaCl solution). After the albumin solution was mixed well with saline solution, the infusion bottle was kept still at horizontal position, and 0.8 ml of the cabazitaxel API solution was injected rapidly into the infusion bottle by injecting underneath the liquid surface of the albumin solution (the injection process took about 1 second). Then the infusion bottle was immediately gently inverted for about 20 times (the inversion process took about 60 seconds), and the solution was mixed well to obtain a transparent solution free of visible particles or precipitates. The particulate matter and pH of the transparent solution were also measured. 1 ml of the transparent solution has about 37 particles that are greater than 10 μm in size and about 7 particles that are greater than 25 μm in size. pH of the transparent solution is 6.91. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 58 particles that are greater than 10 μm in size and about 3 particles that are greater than 25 μm in size.

16 ml of a 20% solution of human serum albumin for infusion (w/v) was added into a 250 ml infusion bottle containing 83 ml 5% dextrose solution. After the albumin solution was mixed well with dextrose solution, the infusion bottle was kept still at horizontal position, and 0.8 ml of the cabazitaxel API solution was injected rapidly into the infusion bottle by injecting underneath the liquid surface of the albumin solution (the injection process took about 1 second). Then the infusion bottle was immediately gently inverted for about 20 times (the inversion process took about 60 seconds), and the solution was mixed well to obtain a transparent solution free of visible particles or precipitates. The particulate matter and pH of the transparent solution were also measured. pH of the transparent solution is 7.07. 1 ml of the transparent solution has about 60 particles that are greater than 10 μm in size and about 13 particles that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 42 particles that are greater than 10 μm in size and about 1 particle that are greater than 25 μm in size.

4 ml of a 20% solution of human serum albumin for infusion (w/v) was added into a 250 ml infusion bottle containing 194 ml normal saline solution (0.9% NaCl solution). After the albumin solution was mixed well with saline solution, the infusion bottle was kept still at horizontal position, and 1.6 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times (the inversion process took about 60 seconds), and the solution was mixed well to obtain a transparent solution free ofvisible particles or precipitates. The particulate matter of the transparent solution was also measured. 1 ml of the transparent solution has about 57 particles that are greater than 10 μm in size and about 1 particle that are greater than 25 μm in size. The solution was kept at 20° C. to 25° C. for 4 hours, which stayed as a transparent solution free of visible particles or precipitates for 4 hours. The particulate matter of the transparent solution was also measured at 4 hour. 1 ml of the transparent solution at 4 hour has about 62 particles that are greater than 10 μm in size and about 3 particles that are greater than 25 μm in size.

5 ml of a 20% solution of human serum albumin for infusion (w/v) was added into a 250 ml infusion bottle containing 193 ml normal saline solution (0.9% NaCl solution). After the albumin solution was mixed well with saline solution, the infusion bottle was kept still at horizontal position, and 1.6 ml of the cabazitaxel API solution was injected rapidly into the infusion bottle by injecting underneath the liquid surface of the albumin solution (the injection process took about 1-2 seconds). Then the infusion bottle was immediately gently inverted for about 20 times (the inversion process took about 60 seconds), and the solution was mixed well to obtain a transparent solution free of visible particles or precipitates. The particulate matter of the transparent solution was also measured. 1 ml of the transparent solution has about 46 particles that are greater than 10 μm in size and about 14 particle that are greater than 25 μm in size. The solution was kept at 20° C. to 25° C. for 3 hours, which stayed as a transparent solution free of visible particles or precipitates for 3 hours. The particulate matter of the transparent solution was also measured at 3 hour. 1 ml of the transparent solution at 3 hour has about 54 particles that are greater than 10 μm in size and about 15 particles that are greater than 25 μm in size.

### Example 15

21.5 mg citric acid was dissolved in 21.5 ml dehydrated ethanol to obtain a citric acid solution (about 1 mg/ml). 399.2 mg of cabazitaxel was dissolved in 39.2 ml of dehydrated ethanol, followed by adding 0.8 ml of the prepared citric acid solution to obtain a cabazitaxel product solution (400 ml) as a clear solution, which was filtered by a 0.22 micron filter to give a clear solution. 3 ml of the filtered product solution was mixed with 3 ml of water to obtain a clear solution, which has pH value about 4.97. Total 15 vials, each with 2 ml of the cabazitaxel product solution, were obtained from the remaining filtered product solution.

### Example 16

5 ml of a 20% solution of human serum albumin for infusion (w/v) was added into a 250 ml infusion bottle containing 94 ml normal saline solution (0.9% NaCl solution). After the albumin solution was mixed well with saline solution, the infusion bottle was kept still at vertical up position, and 1 ml of the cabazitaxel product solution from Example 15 was injected rapidly into the infusion bottle by injecting from the top of the infusion bottle and above the liquid surface of the albumin solution (the injection process took about 1 second). Then the infusion bottle was immediately gently inverted for about 20 times (the inversion process took about 60 seconds). Then a cloudy solution with visible particles and white precipitates was obtained in the infusion bottle.

5 ml of a 20% solution of human serum albumin for infusion (w/v) was added into a 250 ml infusion bottle containing 94 ml normal saline solution (0.9% NaCl solution). After the albumin solution was mixed well with saline solution, the infusion bottle was kept still at vertical up position, and 1 ml of the cabazitaxel product solution from Example 15 was injected into the infusion bottle by injecting from the top of the infusion bottle and above the liquid surface of the albumin solution (the injection process took about 4-5 seconds). Then the infusion bottle was immediately gently inverted for about 20 times (the inversion process took about 60 seconds). Then a cloudy solution with visible particles and white precipitates was obtained in the infusion bottle.

### Example 17

Preparation of Composition A1: 8 ml of a 20% solution of human serum albumin for infusion (w/v) was added into a 250 ml infusion bottle containing 190 ml saline solution (0.9% NaCl solution). After the albumin solution was mixed well with saline solution, the infusion bottle was kept still at horizontal position, and 1.6 ml of a cabazitaxel injection solution with 10 mg/ml cabazitaxel and 0.02 mg/ml citric acid in dehydrated ethanol was injected into the 250 ml infusion bottle by injecting underneath the liquid surface of the albumin-saline solution. After the injection, the solution was mixed well by gently inversions to obtain a clear aqueous solution as composition A1.

Preparation of Composition A2: Prepared by the same procedure as for composition A1 using 29.36 ml saline in 100 ml infusion bottle, 10 ml a 20% solution of human serum albumin for infusion (w/v), and 0.64 ml of a cabazitaxel injection solution with 10 mg/ml cabazitaxel and 0.04 mg/ml citric acid in dehydrated ethanol and PEG 300 (4:1, v/v).

Preparation of Composition A3: Prepared by the same procedure as for composition A1 using 29.52 ml saline in 100 ml infusion bottle, 10 ml a 20% solution of human serum albumin for infusion (w/v), and 0.48 ml of a cabazitaxel injection solution with 10 mg/ml cabazitaxel and 0.04 mg/ml citric acid in dehydrated ethanol and PEG 300 (4:1, v/v).

Preparation of Composition A4: Prepared by the same procedure as for composition A1 using 29.2 ml saline in 100 ml infusion bottle, 10 ml a 20% solution of human serum albumin for infusion (w/v), and 0.8 ml of a cabazitaxel injection solution with 10 mg/ml cabazitaxel and 0.04 mg/ml citric acid in dehydrated ethanol and PEG 300 (4:1, v/v).

Preparation of Composition A5: Prepared by the same procedure as for composition A1 using 91.5 ml saline in 100 ml infusion bottle, 7.5 ml a 20% solution of human serum albumin for infusion (w/v), and 1 ml of a cabazitaxel injection solution with 10 mg/ml cabazitaxel and 0.04 mg/ml citric acid in dehydrated ethanol and PEG 300 (4:1, v/v).

Preparation of Composition A6: Prepared by the same procedure as for composition A1 using 89.8 ml saline in 100 ml infusion bottle, 9 ml a 20% solution of human serum albumin for infusion (w/v), and 1.2 ml of a cabazitaxel injection solution with 10 mg/ml cabazitaxel and 0.04 mg/ml citric acid in dehydrated ethanol and PEG 300 (4:1, v/v).

Preparation of Composition A7: Prepared by the same procedure as for composition A1 using 93.2 ml saline in 100 ml infusion bottle, 6 ml a 20% solution of human serum albumin for infusion (w/v), and 0.8 ml of a cabazitaxel injection solution with 10 mg/ml cabazitaxel and 0.04 mg/ml citric acid in dehydrated ethanol and PEG 300 (4:1, v/v).

Preparation of Composition A8: Prepared by the same procedure as for composition A1 using 96.2 ml saline in 100 ml infusion bottle, 3 ml a 20% solution of human serum albumin for infusion (w/v), and 0.8 ml of a cabazitaxel injection solution with 10 mg/ml cabazitaxel and 0.04 mg/ml citric acid in dehydrated ethanol and PEG 300 (4:1, v/v).

Preparation of Composition A9: Prepared by the same procedure as for composition A1 using 50.1 ml saline in 100 ml infusion bottle, 7.5 ml a 20% solution of human serum albumin for infusion (w/v), and 2.4 ml of a cabazitaxel injection solution with 10 mg/ml cabazitaxel and 0.04 mg/ml citric acid in dehydrated ethanol and PEG 300 (4:1, v/v).

Preparation of Composition A10: Prepared by the same procedure as for composition A1 using 50.7 ml saline in 100 ml infusion bottle, 7.5 ml a 20% solution of human serum albumin for infusion (w/v), and 1.8 ml of a cabazitaxel injection solution with 10 mg/ml cabazitaxel and 0.04 mg/ml citric acid in dehydrated ethanol and PEG 300 (4:1, v/v).

Concentration and percentage of free (unbound in solution) cabazitaxel in compositions A1, A2, A3, A4, A5, A6. A7, A8, A9, and A10 were measured by ultrafiltration through a 30-kDa membrane. After ultrafiltration using centrifuge with a 15 mL-ultrafiltration tube (Millipore. Amicon Ultra-15 30K) containing the composition solution, the filtrate was analyzed for free (unbound in solution) cabazitaxel.

The results of the measurements are shown in Table 2 below. In Table 2, total amount of cabazitaxel and concentration of free (unbound in solution) cabazitaxel in the compositions were measured in ultrafiltration experiments, and amount of albumin in the compositions was calculated based on volume of 20% solution of human serum albumin for infusion (w/v) added and total volume of the compositions.

TABLE 2
Amount of albumin, total amount of cabazitaxel, concentration
of free (unbound in solution) cabazitaxel, and percentage
of free (unbound in solution) cabazitaxel in the compositions

| | | | Concentration | Percentage |
|---|---|---|---|---|
| Com- | Amount of | Total amount | of free (unbound | of free (unbound |
| posi- | Albumin | of cabazitaxel | in solution) | in solution) |
| tion | (w/v) | (mg/ml) | cabazitaxel (mg/ml) | cabazitaxel (%) |
| A1 | 0.8% | 0.092 | 0.052 | 56.9 |
| A2 | 5% | 0.160 | 0.049 | 30.5 |
| A3 | 5% | 0.120 | 0.046 | 38.1 |
| A4 | 5% | 0.191 | 0.044 | 7.8 |
| A5 | 1.5% | 0.105 | 0.052 | 49.4 |
| A6 | 1.8% | 0.122 | 0.055 | 45.1 |
| A7 | 1.2% | 0.077 | 0.047 | 61.5 |
| A8 | 0.6% | 0,075 | 0,040 | 53% |
| A9 | 2.5% | 0.390 | 0.052 | 13% |
| A10 | 2.5% | 0.301 | 0.044 | 15% |

### Example 18

12 ml of a 20% solution of human serum albumin for infusion (w/v) was added into a 250 ml infusion bottle containing 186.4 ml saline solution (0.9% NaCl solution). After the albumin solution was mixed well with saline solution, the infusion bottle was kept still at horizontal position, and 1.6 ml of a cabazitaxel injection solution with 10 mg/ml cabazitaxel and 0.04 mg/ml citric acid in dehydrated ethanol and PEG 300 (4:1, v/v) was injected into the 250 ml infusion bottle by injecting underneath the liquid surface of the albumin-saline solution. After the injection, the solution was mixed well by gently inversions to obtain a clear aqueous solution as composition B.

The compositions of B was kept at room temperature for 2 hours. At initial, 1 hour, and 2 hour, concentration of free (unbound in solution) cabazitaxel in the compositions (aqueous solutions) was measured by ultrafiltration through a 30-kDa membrane. The results were shown in Table 3. The data form Table 3 indicated that there was no substantial change for the concentration of free (unbound in solution) cabazitaxel in the composition of B, when kept at room temperature for 2 hours.

TABLE 3
Concentration and percentage of free (unbound in solution)
cabazitaxel in the composition B at initial, 1 hour, and 2 hour

| | Time | Concentration of free | Percentage of free |
|---|---|---|---|
| | Point | (unbound in solution) | (unbound in solution) |
| Composition | (hour) | cabazitaxel (mg/ml) | cabazitaxel (%) |
| Composition | 0 | 0.047 | 60.3 |
| B | 1 | 0.046 | 59.0 |
| | 2 | 0.047 | 60.3 |

### Example 19

A pharmacokinetics (PK) study was conducted to investigate cabazitaxel levels in plasma after a single intravenous administration of composition comprising cabazitaxel and human serum albumin at a dose of 2 mg/kg in rats. The composition used in the PK study was prepared using the same procedure for preparing composition B used in Example 18 to obtain the composition as a clear aqueous solution.

In the PK study, six adult male Sprague-Dawley rats were administered with the composition solution obtained in the study at a dose of 2 mg/kg following a 20-minute intravenous infusion. Approximately 0.15 mL blood samples were collected at at 0 (pre-dose), 0.167 (10 min), 0.25 (15 min), 0.317 (19 min, one minute prior to the end of infusion), 0.367 (22 min), 0.5 (30 min), 0.833 (50 min), 1.33 (80 min), 2.33 (140 min), 5.33 (320 min), 10.33 (620 min) and 24.33 hours (1460 min) post-dose from each study animal. Plasma samples were prepared and kept at freezer for bioanalysis. The concentrations of cabazitaxel in plasma were determined by liquid chromatography tandem mass spectrometry (LC-MS/MS) method. The linearity was evaluated at the linear range of 2.00-2000 ng/mL for Cabazitaxel.

After a single intravenous infusion dose of the composition solution at a dose of 2 mg/kg, cabazitaxel was able to be detected in plasma until the last blood collection time (24 h post the end of infusion). The mean plasma pharmacokinetics parameters are shown in Error! Reference source not found. The semi-logarithmic mean plasma concentration-time profiles are illustrated in Error! Reference source not found.

TABLE 4
Mean pharmacokinetic parameters of cabazitaxel in rats administered
composition solution following a single intravenous infusion

| PK Parameter | Mean | SD |
|---|---|---|
| Cₘₐₓ (ng/ml) | 449 | 115 |
| Tₘₐₓ (h) | 0.281 | 0.0618 |
| T_{1/2}(h) | 5.25 | 1.66 |
| Vdₛₛ(L/kg) | 27.3 | 9.83 |
| CL(mL/min/kg) | 164 | 29.7 |
| AUC₀₋ₗₐₛₜ (h · ng/mL) | 190 | 45.7 |
| AUC_{0-inf} (h · ng/mL) | 210 | 43.8 |

### Example 20

12.22 mg of citric acid and 3.069 g of cabazitaxel were dissolved with 71.11 g of dehydrated ethanol in a 250 ml beaker. Then the solution was transferred into a 1 L beaker using 54.29 g, 65.32 g, and 46.18 g of dehydrated ethanol to rinse the 250 ml beaker three times into the 1 L beaker. The resulting solution was mixed well and filtered by 0.22 micron PTFE filters to give a cabazitaxel product solution. About 10 ml of the product solution was used for quality control analysis. The remaining cabazitaxel product solution was filled into 10 ml vials, and total 143 vials were obtained with 2 ml solution in each vial. The pH of the cabazitaxel product solution obtained is about 4.4. The purity of the cabazitaxel product solution is 99.70%, and the assay of the cabazitaxel product solution is 9.93 mg/ml.

### Example 21

4.258 g of cabazitaxel was dissolved in 66.29 g of dehydrated ethanol in a 250 ml beaker, 16.7 mg citric acid was dissolved in 10.65 g of dehydrated ethanol in a 50 ml beaker, and 94.54 g PEG300 was mixed well with 66.28 g of dehydrated ethanol in a 250 ml beaker. Then the prepared cabazitaxel solution was transferred into a 2 L beaker using 66.29 g of dehydrated ethanol to rinse the 250 ml beaker three times into the 2 L beaker. The PEG300-ethanol solution was transferred into the 2 L beaker using 50.39 g of dehydrated ethanol to rinse the 250 ml beaker two times into the 2 L beaker when stirring. The citric acid solution was transferred into the 2 L beaker using 5.38 g of dehydrated ethanol to rinse the 50 ml beaker into the 2 L beaker when stirring. About 12 ml of the product solution obtained was used for quality control analysis. The remaining solution was filtered by 0.22 micron PTFE filters to give a cabazitaxel product solution. The cabazitaxel product solution was filled into 10 ml vials, and total 132 vials were obtained with 3 ml solution in each vial. The pH of the cabazitaxel product solution obtained is about 5.0. The purity of the cabazitaxel product solution is 99.63%, and the assay of the cabazitaxel product solution is 10.02 mg/ml.

### Example 22

12 ml of a 20% solution of human serum albumin for infusion (w/v) was added into a 250 ml infusion bottle containing 186 ml normal saline solution (0.9% NaCl solution). After the albumin solution was mixed well with saline solution, the infusion bottle was kept still at horizontal position, and 1.6 ml of the cabazitaxel product solution prepared in Example 20 was injected rapidly into the infusion bottle by injecting underneath the liquid surface of the albumin solution (the injection process took about 2 seconds). Then the infusion bottle was immediately gently inverted for about 20 times (the inversion process took about 1 minute), and the solution was mixed well to obtain a transparent solution free of visible particles or precipitates. The particulate matter of the transparent solution was also measured. 1 ml of the transparent solution has about 20 particles that are greater than 10 μm in size and about 4 particles that are greater than 25 μm in size. The solution was kept at 20° C. to 25° C. for 7 hours, which stayed as a transparent solution free of visible particles or precipitates for 7 hours. The particulate matter of the transparent solution was also measured at 7 hour. 1 ml of the transparent solution at 7 hour has about 64 particles that are greater than 10 μm in size and 7 particles that are greater than 25 μm in size.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-31. (canceled)

32. An infusion composition comprising cabazitaxel and human serum albumin contained in an infusion bag/bottle, wherein the infusion composition is prepared by injecting a liquid composition comprising cabazitaxel and ethanol into an infusion bag or bottle containing an aqueous composition comprising human serum albumin in a parenterally acceptable vehicle, wherein mixing or agitation is not needed in the injection process, wherein the mixing of the liquid composition and the aqueous composition is done in an infusion bag/bottle, wherein the infusion composition is prepared in less than 24 hours prior to being infused or administered to patients, and wherein the liquid composition comprising cabazitaxel and ethanol is a sterile solution.

33. The infusion composition of claim 32, wherein the infusion composition comprises from about 5 mg to about 60 mg of cabazitaxel, from about 0.5 ml to about 15 ml of ethanol, and from about 0.5 g to about 20 g of human serum albumin.

34. The infusion composition of claim 32, wherein the infusion composition is prepared in a hospital or clinic prior to being infused or administered to patients.

35. The infusion composition of claim 32, wherein the aqueous composition comprising human serum albumin is prepared by adding a clinically-used solution of human serum albumin for infusion into an infusion bag or bottle which contains a parenterally acceptable vehicle.

36. The infusion composition of claim 32, wherein at least 10% cabazitaxel in the infusion composition is Free (unbound in solution) cabazitaxel.

37. The infusion composition of claim 32, wherein at least 30% cabazitaxel in the infusion composition is Free (unbound in solution) cabazitaxel.

38. The infusion composition of claim 32, wherein after the injection is completed, the infusion bag or bottle is inverted for from 10 seconds to 5 minutes to mix well of the liquid composition and the aqueous composition.

39. The infusion composition of claim 32, wherein the infusion composition is a transparent infusion solution free of visible particles or precipitates.

40. The infusion composition of claim 39, wherein the infusion composition stays transparent free of visible particles or precipitates for at least about 2 hours.

41. The infusion composition of claim 39, wherein the concentration of cabazitaxel in the infusion composition is from about 0.05 mg/ml to about 0.2 mg/ml.

42. The infusion composition of claim 39, wherein the amount of human serum albumin in the infusion composition of cabazitaxel is from about 0.1% (w/v) to about 5% (w/v).

43. The infusion composition of claim 39, wherein the pH value of the infusion composition is from about 4 to about 9.5.

44. The infusion composition of claim 39, wherein 1 ml of the infusion composition comprises no more than 50 particles that are greater than 10 μm in size.

45. The infusion composition of claim 39, wherein 1 ml of the infusion composition comprises no more than 100 particles that are greater than 10 m in size.

46. The infusion composition of claim 39, wherein 1 ml of the parenteral infusion composition comprises no more than 10 particles that are greater than 25 μm in size.
